(19) United States
(12) Patent Application Publication
(10) Pub. No.: US 2013/0261670 A1
(43) Pub. Date: Oct. 3, 2013

(54) IMPLANT AND ASSOCIATED INSTRUMENTS AND METHODS

(71) Applicant: DEPUY SYNTHES PRODUCTS, LLC, Raynham, MA (US)

(72) Inventors: Bruno LAENG, Oberdorf (CH); Philippe GEDET, Oberdorf (CH)

(21) Appl. No.: 13/795,114

(22) Filed: Mar. 12, 2013

Related U.S. Application Data
(60) Provisional application No. 61/617,413, filed on Mar. 29, 2012.

(51) Int. Cl. A61B 17/80

(52) U.S. Cl. CPC A61B 17/8004
USPC 606/281; 606/282

(57) ABSTRACT

A device for bone joint distraction includes a first bone plate for insertion on a first side of the bone joint, a second bone plate for insertion on a second side of the bone joint, a first elongated rod insertable into the bone joint, wherein the first bone plate is non-rotatably coupled to the first rod, and a second elongated rod insertable into the bone joint, wherein the second bone plate is rotatably coupled to the second rod, the first and second bone plates being movable relative to one another within a predetermined range of movement in a plane including longitudinal axes of the first and second rods to distract the joint.

### PRIORITY CLAIM

This application claims priority to U.S. Provisional Application Ser. No. 61/617,413 filed on Mar. 29, 2012 and entitled “Implant and Associated Instruments and Methods,” the entire disclosure of which is incorporated herein by reference.

### FIELD OF THE INVENTION

The present invention generally relates to a device for the treatment of a bone joint and to methods for use of the same. More particularly, the present invention relates to a device for the distraction of a bone joint and to a method for use of the same. Embodiments of the invention relate to a subcutaneously implantable device for the distraction of a bone joint, to a kit for assembling a device according to the invention, to a method for the distraction of a knee joint and to a method for the treatment of knee joint-arthritis.

### BACKGROUND

Joint distraction in the treatment of severe ankle and hip osteoarthritis has demonstrated good clinical outcomes. It has been shown that an external rigid fixation and a joint distraction of 5 mm. over a period of two to three months is beneficial to both the knee joint and the ankle joint. The knee joint can be thought of as having two different types of motion. The first type is a rotation of the femur relative to the tibia. The second type is a sliding motion, where after or simultaneously to the rotation, the femur slides relative to the tibia. These motions become difficult when cartilage has been damaged.

It is therefore an objective of the invention to provide a device for the distraction of bone joints which is implantable into the human or animal body and which fully relieves the bone joint, particularly the knee joint from stresses in a sufficient range of motion to therewith achieve a regeneration of the tissue, particularly of the cartilage tissue while further permitting a limited motion of the bone joint.

### SUMMARY OF THE INVENTION

The present invention is directed to a device for bone joint distraction, comprising a first bone plate for insertion on a first side of the bone joint, a second bone plate for insertion on a second side of the bone joint, a first elongated rod insertable into the bone joint, wherein the first bone plate is non-rotatably coupled to the first rod, and a second elongated rod insertable into the bone joint, wherein the second bone plate is rotatably coupled to the second rod, the first and second bone plates being movable relative to one another within a predetermined range of movement in a plane including longitudinal axes of the first and second rods to distract the joint.

The present invention relates to a device for the distraction of a bone joint comprising: A) a first rod shaped member extending along a longitudinal axis from a first end to a second end; B) a second rod shaped member extending from a first end to a second end along a longitudinal axis parallel to and a spaced Z>0 from the longitudinal axis of the first rod shaped member; C) a first connecting member connectable to the first ends of the first and second rod shaped members; and D) a second connecting member connectable to the second ends of the first and second rod shaped members, wherein E) the first and second ends of the second rod shaped member are joinable to the two connecting members by means of an articulation, so that the two connecting members are rotatable about the longitudinal axis of the second rod shaped member, and wherein F) each of the first and second connecting members is reversibly fixable to one of the first and second ends of the first rod shaped member.

Advantages of the device according to the invention include that: (1) the first rod shaped member which is fixed in the tibia together with the first and second connecting members forms a rigid support not moveable relative to the tibia so that only the second rod shaped member which is fixed to the femur is rotatable relative to the support via the articulations. Thus, a hinge joint for the femur is formed which is rigidly fixed in the tibia. This configuration of the device permits the establishment of a fixed spacing between the sliding surfaces of the femur and the tibia so that the knee joint is distracted and fully unloaded in a certain range of motion; (2) the second rod shaped member defines a rotational axis of the knee joint which is fixedly positioned at the bone joint. The rotational axis of the knee joint which coincides with the longitudinal axis of the second rod shaped member implanted in the femur can thus be approximately aligned with the natural rotational axis of the femur for a certain angular range up to 30°; (3) only a rotation of the bones adjoining the bone joint relative to each other about the rotational axis is permitted while the relative sliding motion of the two joint surfaces can be avoided during a period of 2 to 3 months in which the device is implanted; (4) the knee distractor allows for unloading of the knee joint, which allows for regeneration of cartilage-like matter on the surface of the femur condyles and the tibial plateau; (5) a postponement for a knee joint replacement for about ten years can be achieved; and (6) the device can be subcutaneously affixed to the knee joint.

The device according to the invention is preferably configured for a treatment of the knee joint but can be applied in other embodiments to the elbow joint as well.

Further advantageous embodiments of the invention can be commented as follows:

In a special embodiment of the device each of the first and second connecting members comprises a longitudinal axis and the first and second connecting members are incompressible in the direction of their longitudinal axes. This configuration of the device offers the advantage that the compressive forces between the femur and the tibia, in particular, the femoral condyles and the tibial plateau, are transmitted completely through the connecting members so that the compressive forces are not exerted onto the knee joint itself.

In a further embodiment of the device the first and second connecting members are each rotatively form-fittingly fixable to one of the first and second ends of the first rod shaped member. The rotatively form-fitting fixation of the two connecting members at the ends of the first rod shaped member results in a stable positioning of the articulations and the second rod shaped member relative to the tibia.

In a further embodiment the device comprises reversibly securable adjusting means and the spacing Z is adjustable and reversibly fixable by means of the adjusting means. By means of the adjusting means arranged at the connecting members and the first rod shaped member the knee joint can be distracted and retained in a distracted state. Ideally, the distraction of the knee joint amounts to about 5 mm. The articulating surfaces of the tibia and the femur can thus be retained in a selected and fixed spacing relative to each other so that the knee joint is fully relieved of the load in the complete range of motion. To achieve a distraction of the articulating surfaces of about 5 mm and having the first and second rod shaped members properly positioned in the femur and the tibia the spacing Z can be in a range between 30 mm and 40 mm.

In another embodiment of the device the first and second rod shaped member each comprise a peripheral surface including anti-rotation means. By this means the first and second rod shaped members are rigidly secured in the bone with respect to rotation about their longitudinal axes.

In another embodiment of the device the anti-rotation means each comprise at least one ridge, preferably in the form of a rib or fin extending essentially parallel to the longitudinal axis of the first and second rod shaped member. This configuration of the anti-rotation means allows the advantage that the two rod shaped members can be easily driven into the bores in the tibia and in the femur by keeping the desired orientation.

In a further embodiment of the device the first and/or second rod shaped member comprise a cannulation. Through the cannulations each a pin can be inserted, so that the two pins can be linked to a distraction instrument. By this means the spacing between the first and second rod shaped member can be adjusted with the help of a distraction instrument and fixed through tightening of the screw connection between the first rod shaped member and the connecting members.

In a further embodiment of the device the first and second connecting member each comprises a longitudinal axis and the adjusting means each comprise a longitudinal recess or slot arranged in the first and second connecting member and extending in the direction of the longitudinal axis of the first and second connecting member.

In again a further embodiment of the device the first rod shaped member comprises a peripheral surface which is provided with flattenings at the first and second end. Therewith the advantage of a simple configuration of the joining between the first rod shaped member and the connecting members can be achieved. The flattenings can be positioned in the longitudinal recesses in the connecting members rotationally stable but displaceable parallel to the longitudinal axes of the connecting members.

In yet a further embodiment of the device the adjusting means each comprise a reversibly fixable securing means for securing the first and second connecting member each to one end of the first rod shaped member.

In another embodiment of the device the first and second connecting member comprise a toothing in the area of the longitudinal recesses or slots and the securing means are provided with a corresponding toothing. Thus, the first rod shaped member cannot move relative to the connecting members even when a high load is applied onto the knee joint, so that the spacing Z between the longitudinal axes of the first and second rod shaped members remains constant.

In again another embodiment of the device each articulation is configured as a hinge joint comprising a hinge pin including a cylindrical portion pivotably received in a bore in each one of the first and second connecting members, a head with a diameter larger than the diameter of the cylindrical portion and a threaded portion with a diameter smaller than the diameter of the cylindrical portion and the threaded portion of each hinge pin is screwable into one end of the second rod shaped member. This configuration allows the advantage that the connecting members are each retained between a first shoulder formed on the hinge pin at the transition between the head and the cylindrical portion and an end of the second rod shaped member.

In a further embodiment of the device each of the first and second connecting members is mounted on the hinge pin with an axial clearance S in the direction of the longitudinal axis of the second rod shaped member. Due to this axial clearance S the friction of the hinge joints formed between the second rod shaped member and the connecting members can be significantly reduced.

In still a further embodiment of the device each articulation is configured as a hinge joint and comprises sliding surfaces, which are preferably provided with a CrCo, TiN or ADLC (amorphous diamond-like carbon) coating.

According to a further aspect of the invention, there is provided a method for the distraction of knee joints, the method comprising the steps of: providing a device according to the invention; and distracting a knee joint using the device.

According to again a further aspect of the invention, there is provided a method for the treatment of knee joint-arthritis, the method comprising the steps of: providing a device according to the invention; and treating the knee joint-arthritis using the device.

In accordance with another aspect of the invention, a method is provided for knee joint distraction using a device according to the invention and characterized by the steps: a) determining a center of rotation of a knee joint; b setting a k-wire in the center of rotation; c) performing at least a lateral and a medial incision in the area of the knee joint; d) drilling a bore hole along the k-wire in the femur, the bore hole extending in the direction of the transverse axis of a patient; e) drilling a bore hole along the k-wire in the tibia, the bore hole extending in the direction of the transverse axis of a patient, preferably, the bore hole is drilled parallel to the bore hole in the femur; f) beating the first rod shaped member into the bore hole in the tibia; g) beating the second rod shaped member into the bore hole in the femur; h) mounting and securing the first connecting member to the first end of the second rod shaped member and the second connecting member to the second end of the second rod shaped member; i) pre-mounting each a securing means to the first and second end of the first rod shaped member without fixing the first and second connecting member to the first rod shaped member through tightening the securing means; j) distracting of the knee joint by means of a distractor instrument, preferably in a step-by-step manner; k) fixing the first connecting member to the first end of the first rod shaped member and the second connecting member to the second end of the first rod shaped member by tightening the securing means; l) removing the distractor instrument; and m) closing the incisions.

The method permits the advantage that by means of the two connecting members and the first rod shaped member fixed in the tibia a rigid support is produced for the second rod shaped member which is rotatable with respect to the two connecting members and which is fixed in the femur. The second rod shaped member is thus rotatable relative to the support fixed in the tibia, so that the rotational axis of the second rod shaped member and consequently the rotational axis of the femur can be located as close as possible to the natural rotational axis of the femur. The device is thus configured as a hinge joint for the femur.

In a special embodiment of the method the bore hole in the tibia and the bore hole in the femur are drilled with their hole axes arranged with a spacing Z>0, preferably amounting to between 30 mm and 60 mm.

In a further embodiment of the method the bore hole in the tibia is drilled orthogonally to the longitudinal axis of the tibia.

In a further embodiment of the method the bore hole in the femur is drilled orthogonally to the longitudinal axis of the femur.

In another embodiment of the method the knee joint is distracted in step up to a 5 mm distraction. For example, the distraction could be in 1 mm steps to a distraction of up to 5 mm. The distraction may be 1 mm during a set period of time. For example, the set period of time could be a 1 mm distraction every 5 minutes until the 5 mm distraction is achieved. The step-wise distraction may allow the ligaments to stretch over a period of time and may minimize damage to the ligament.

In another embodiment the method comprises the additional step of rotatively aligning the first rod shaped member so that each two parallel flattenings extend essentially parallel to each a line connecting the longitudinal axes at the first and second ends of the first and second rod shaped members.

In a further embodiment the method comprises the further step of inserting each a pin through the cannulations in the first and second rod shaped member so that a distractor instrument can be attached to the pins once the device is pre-assembled.

In again a further embodiment the method includes the additional steps: performing at least a lateral and a medial incision at a knee joint into which a device has been previously implanted; explanting the device from the knee joint; and closing the incisions.

In another embodiment of the method the explantation of the device is performed 2 to 3 months after implanting the device into the knee joint.

In yet another embodiment of the method each a lateral and a medial puncture incision in the area of the knee joint is performed for insertion of the first and second rod shaped member. By this means a minimal invasive implantation of the device can be performed.

In a further embodiment of the method the first and second connecting member are implanted via the puncture incisions performed for insertion of the first and second rod shaped member.

In another embodiment of the method each an incision traversing the knee joint laterally and medially is performed.

In again another embodiment of the method the bore hole in the femur drilled under step c) has a bore axis which passes through the femur in the area of the natural center of rotation of the knee joint.

In yet another embodiment of the method the distractor instrument is attached to the device by performing the steps of: inserting a pin each in the cannulation of the first and second rod shaped member; and mounting the distractor instrument to the pins.

In a further embodiment of the method the device is subcutaneously implanted.

In accordance with a further aspect of the invention, a kit is provided for assembling a device according the invention, the kit comprising: a first rod shaped member with a longitudinal axis, a first end and a second end; a second rod shaped member with a first end, a second end and a longitudinal axis; a first connecting member which is connectable to the first ends of the first and second rod shaped member; and a second connecting member which is connectable to the second ends of the first and second rod shaped member, wherein the second rod shaped member is mountable to the first and second connecting members with its longitudinal axis essentially parallel to and at a spacing Z>0 apart from the longitudinal axis of the first rod shaped member; the first and second end of the second rod shaped member are joinable to the two connecting members by means of an articulation, so that the two connecting members are rotatable about the longitudinal axis of the second rod shaped member; and the first and second connecting member are each reversibly fixable to one of the two ends of the first rod shaped member.

In a special embodiment of the kit each of the first and second connecting members comprises a longitudinal axis and the first and second connecting members are incompressible in the direction of their longitudinal axes. The compressive forces between the femur and the tibia are thus transmitted through the connecting members so that the compressive forces are not exerted onto the articulation surfaces of the knee joint itself.

In a further embodiment of the kit each of the first and second connecting members is rotatively form-fittingly fixable to one of the two ends of the first rod shaped member.

In a further embodiment the kit comprises reversibly securable adjusting means and the spacing Z is adjustable and reversibly fixable by means of the adjusting means.

In another embodiment of the kit the first and second rod shaped member each comprise a peripheral surface including anti-rotation means.

In another embodiment of the kit the anti-rotation means each comprise at least one ridge, preferably in the form of a rib or fin extending essentially parallel to the longitudinal axes of the first and second rod shaped member.

In again another embodiment of the kit the first and/or second rod shaped member comprise a cannulation.

In yet another embodiment of the kit the first and second connecting member each comprises a longitudinal axis and wherein the adjusting means each comprise a longitudinal recess or slot arranged in the first and second connecting member and extending in the direction of the longitudinal axis of the first and second connecting member.

In a further embodiment of the kit the first rod shaped member comprises a peripheral surface which is provided with flattenings at the first and second end.

In again a further embodiment of the kit the adjusting means each comprise a reversibly fixable securing means for securing the first and second connecting member each at one end of the first rod shaped member.

In again a further embodiment of the kit the first and second connecting member comprise a toothing in the area of the longitudinal recesses or slots and the securing means are provided with a corresponding toothing.

In another embodiment of the kit each articulation is configured as a hinge joint comprising a hinge pin including a cylindrical portion pivotably received in a bore in each one of the first and second connecting member, a head with a diameter larger than the diameter of the cylindrical portion and a threaded portion with a diameter smaller than the diameter of the cylindrical portion and the threaded portion of each hinge pin is screwable into one end of the second rod shaped member.

According to a further aspect of the invention, there is provided a method for temporarily implanting a hinge joint implant, the method comprising: providing a kit according the invention; and implanting a device according to the invention using the kit.

According to a still further aspect of the invention, there is provided an implant assembly. The implant assembly has a first implant, a second implant and a connector. Both the first implant is non-rotatably coupleable to the connector and the second implant is rotatably coupleable to the connector at locations spaced apart in a displacement direction at a defined displacement.

The implant assembly may be a distractor. The distractor may be arranged to distract a joint in a human or animal body. The distractor may have a first implant non-rotatably coupled to a connector and may have a second implant rotatably coupled to the connector. The first implant and second implant may be coupled to the connector at locations spaced apart in a displacement direction and at a desired distraction displacement.

The connector may have a first connector and a second connector. The first connector may be configured to engage first ends of the first and second implants. The second connector may be configured to engage second ends of the first and second implants.

The second implant may be rotatably coupled to the connector with at least one fastener. The fastener may have a shaft defining an axis of rotation of the second implant relative to the connector. A fitting portion of the shaft may be dimensioned to form fit into an aperture in the connector for defining the axis of rotation. The fitting portion may extend from a head portion shaped to abut the connector, the shaft further comprising a threaded portion extending from the fitting portion, the threaded portion arranged to couple the fastener to the second implant.

The implant assembly may have a spacing arrangement in a first zone and a rotation arrangement in a second zone. The spacing arrangement may be for setting the displacement between the first and the second implants. The first zone may be spaced apart from the second zone in the displacement direction. The first implant may be non-rotatably coupleable to the connector in the first zone using the spacing arrangement and the second implant may be rotatably coupleable to the connector in the second zone using the rotation arrangement. The spacing arrangement may have at least a first spacer selectively engageable with a second spacer to set the displacement between the first and the second implants. The second spacer may have an engagement region in the first zone arranged to receive and retain an engagement portion of the first spacer at a selected engagement point for setting the displacement between the first and the second implants. The engagement region may have a holding array for receiving and retaining the engagement portion at discrete points in the displacement direction to set the defined displacement between the first and the second implants. The holding array may have a first series of ridges for receiving and retaining the engagement portion and the engagement portion has a second series of ridges shaped to complement the first series of ridges.

The connector may include the second spacer. The first spacer may have a washer-like element and a fastener. The washer-like element may have an aperture for receiving the fastener therethrough. An engagement surface may be provided that features the engagement portion. The fastener may have a threaded surface for coupling with an end of one of the first or second implants.

The spacing arrangement may have a slot arranged in the displacement direction. A portion of an external surface of the first implant may be slideable within the slot. This may be for setting the displacement between the first and the second implants. A portion of an external surface of the first implant may interlock with a slot in the connector for preventing rotation of the first implant relative to the connector. The slot may have a first sidewall spaced apart from a second sidewall. The first and second sidewalls may be planar and aligned with the displacement direction. A portion of an external surface of the first implant may have first and second planar surfaces. The first planar surface may be arranged to abut the first sidewall and the second planar surface may be arranged to abut the second sidewall. The abutment may prevent rotation. The abutment may facilitate guiding the sliding of the first implant relative to the connector in a displacement direction.

One or both of the first and second implants may be configured to prevent their rotation relative to a bone, in use. One or both of the first and second implants may have a protrusion on an outer surface. The protrusion may be for engagement with a bone, in use. The protrusion may be for preventing the rotation of the first and/or second implants relative to the bone and for indicating an alignment direction and keeping the desired orientation of the first and second implants when they are being inserted into the joint.

The first and/or the second implants may be substantially elongate and/or rod-like.

The first and/or the second implants may be cannulated.

The connector may be plate-like.

In a yet another aspect of the present invention, there is provided an instrument for setting a displacement between a first and a second implant. The instrument may have a first implant holder adapted to hold a first implant, a second implant holder adapted to hold a second implant, and a spacing arrangement. The spacing arrangement may have a displacer arranged to vary a displacement between the first implant holder and the second implant holder to set a desired displacement. The spacing arrangement may have also a spacer arranged to hold the first implant holder spaced apart from the second implant holder at the desired displacement.

The instrument may be a distraction setting instrument. The distraction setting instrument may for setting a desired distraction displacement between the first and second implant holders.

The first implant holder may be pivotally connected to the second implant holder. The first implant holder and second implant holder may be pivotally connected in a scissor-like arrangement. In the scissor arrangement, first ends of the first and second implant holder may be adapted to hold the first and second implants, respectively. The spacing arrangement may be arranged between opposed second ends of the implant holders for spacing apart and holding the second ends of the first and second implant holders.

The scissor-like arrangement may have a first and a second scissor-like member. The first scissor-like member may be spaced apart from the second scissor-like member by an axis to which the first and second scissor-like members are pivotally connected. Opposed second ends of the first and second scissor-like members of the first implant holder may be rotatably connected to a first axis and opposed second ends of the first and second scissor-like members of the second implant holder may be rotatably connected to a second axis. The spacing arrangement may be connected between the first and second axes.

The displacer and spacer may be provided by a shaft in combination with a bolt-like element. The shaft may have an external thread and be arranged to engage the first and second implant holders. The bolt-like element may have an internal thread and may be arranged on the shaft. The bolt-like element may be rotatable to engage with the first implant holder to vary the displacement. The external and internal threads may be adapted to abut each other to hold the first implant holder spaced apart from the second implant holder at the desired displacement. The shaft may be fixedly arranged on the first rods and may pass through a channel in the second rod. The bolt-like element may be rotatable to engage with the second rod to vary the displacement to set the desired displacement.

In a still further aspect of the present invention, there is provided a method of distracting a joint. The joint may have a first joint member and a second joint member. The method may include the steps of: arranging a first implant in the first joint member; arranging a second implant in the second joint member; engaging a first connector with first ends of the first and second implants and a second connector with opposed second ends of the first and second implants; engaging first and second fixation elements with the first and second connectors, respectively, at locations adjacent the first implant; engaging third and fourth fixation elements with the first and second connectors, respectively, at locations adjacent the second implant; spacing apart the first and second implants at a desired displacement with a displacement setting instrument; rotatably fixing the first implant to the first and second connectors with the first and second fixation elements; non-rotatably fixing the second implant to the first and second connectors with the third and fourth fixation elements; and removing the displacement setting instrument leaving the first and second implants fixed to the connector at locations spaced apart in a displacement direction and at a desired distraction displacement.

The method may further include the steps of passing a first elongate element in a channel in the first implant and a second elongate element in a channel in the second implant; positioning a first connector over first ends of the first and second elongate elements and a second connector over opposed second ends of the first and second elongate elements; sliding the first fixation element over the first end of the first elongate element and the second fixation element over an opposed second end the first elongate element to engage the first and second connectors, respectively; sliding the third fixation element over the first end of the second elongate element and the fourth fixation element over an opposed second end the second elongate element to engage the first and second connectors, respectively; arranging a first implant holder of the displacement setting tool to engage with the first elongate element and a second implant holder of the displacement setting tool to engage with a second elongate element to hold the first and second implants spaced apart at the desired distraction displacement; rotatably fixing the first implant and non-rotatably fixing the second implant to the first and second connectors respectively at the desired distraction displacement with the first, second, third and fourth fixation elements; and removing the displacement setting instrument and the first and second elongate elements.

The step of arranging a first implant in the first joint member may include the steps of: finding an axis of rotation of the first joint; inserting a guide wire through the first joint along the axis of rotation; drilling a first channel in the first joint using the guide wire for guidance; and inserting the first implant into the first channel.

The step of arranging a second implant in the second joint member may include the steps of: inserting a guide wire through the second joint member at a location determined to provide a desired stability to the first implant; drilling a second channel in the second joint using the guide wire for guidance; and inserting the second implant into the second channel.

The method may further include a step of aligning the first implant with the second implant before engaging the first and second connectors.

### BRIEF DESCRIPTION OF THE DRAWINGS

Several embodiments of the invention will be described in the following by way of example and with reference to the accompanying drawings in which:

Embodiments of the invention will be described in the following by way of example and with reference to the accompanying drawings in which:

FIG. 1 illustrates a perspective view of an embodiment of a device according to the invention;

FIG. 2 illustrates a magnified sectional view of the articulation between the second rod shaped member and the second connecting member of the embodiment of the device according to the invention of FIG. 1;

FIG. 3 illustrates a magnified sectional view of FIG. 2;

FIG. 4 illustrates a perspective plan view of an embodiment of an instrument for use with the device shown in FIGS. 1 to 3;

FIG. 5 illustrates another perspective view of the embodiment of the instrument shown in FIG. 4 in use with the embodiment of the device shown in FIGS. 1 to 3;

FIG. 6 illustrates yet another perspective view of the embodiment of the instrument shown in FIG. 4 in use with the embodiment of the device shown in FIGS. 1 to 3;

FIG. 7 illustrates still another perspective view of the embodiment of the instrument shown in FIG. 4 in use with the embodiment of the device shown in FIGS. 1 to 3;

FIG. 8 illustrates a perspective view of a device according to an exemplary embodiment of the invention;

FIG. 9 illustrates a magnified sectional view of the articulation between the second rod shaped member and the second connecting member of the embodiment of the device of FIG. 8;

FIG. 10 illustrates a magnified sectional view of FIG. 9;

FIG. 11 illustrates a perspective plan view of an embodiment of an instrument for use with the device of FIGS. 8 to 10;

FIG. 12 illustrates another perspective view of the instrument of FIG. 11 in use with the device of FIGS. 8 to 10;

FIG. 13 illustrates another perspective view of the instrument of FIG. 11 in use with the device of FIGS. 8 to 10;

FIG. 14 illustrates another perspective view of the instrument of FIG. 11 in use with the device of FIGS. 8 to 10;

FIG. 15 illustrates a first cross-sectional view of a device according to a second embodiment of the invention;

FIG. 16 illustrates a second cross-sectional view of the device of FIG. 15;

FIG. 17 illustrates a first perspective view of the device of FIG. 15;

FIG. 18 illustrates a second perspective view of the device of FIG. 15;

FIG. 19 illustrates a third cross-sectional view of the device of FIG. 15;

FIG. 20 illustrates a first perspective view of a first rod for the device of FIG. 15;

FIG. 21 illustrates a second perspective view of the first rod of FIG. 20;

FIG. 22 illustrates a cross-sectional view of the first rod of FIG. 20;

FIG. 23 illustrates a perspective view of a first hinge pin for the device of FIG. 15;

FIG. 24 illustrates a cross-sectional view of the first hinge pin of FIG. 23;

FIG. 25 illustrates a first perspective view of a second rod for the device of FIG. 15;

FIG. 26 illustrates a cross-sectional view of the second rod of FIG. 25;

FIG. 27 illustrates a second perspective view of the second rod of FIG. 25;

FIG. 28 illustrates a first perspective view of a blade for the device of FIG. 15;

FIG. 29 illustrates a cross-sectional view of the blade of FIG. 28;

FIG. 30 illustrates a first side view of the blade of FIG. 28;

FIG. 31 illustrates a second side view of the blade of FIG. 28;

FIG. 32 illustrates a perspective view of a second hinge pin for the device of FIG. 15;

FIG. 33 illustrates a cross-sectional view of the second hinge pin of FIG. 32;

FIG. 34 illustrates a perspective view of a washer for the device of FIG. 15;

FIG. 35 illustrates a first side view of the washer of FIG. 34;

FIG. 36 illustrates a second side view of the washer of FIG. 34;

FIG. 37 illustrates a third side view of the washer of FIG. 34;

FIG. 38 illustrates a perspective view of a lateral bone plate for the device of FIG. 15;

FIG. 39 illustrates a first side view of the lateral bone plate of FIG. 38;

FIG. 40 illustrates a second side view of the lateral bone plate of FIG. 38;

FIG. 41 illustrates a perspective view of a medial bone plate for the device of FIG. 15;

FIG. 42 illustrates a first side view of the medial bone plate of FIG. 41;

FIG. 43 illustrates a second side view of the medial bone plate of FIG. 41;

FIG. 44 illustrates a perspective view of a distraction tool according to another embodiment of the invention;

FIG. 45 illustrates a cross-sectional view of the tool of FIG. 44;

FIG. 46 illustrates a perspective view of an end cap for the tool of FIG. 44;

FIG. 47 illustrates a cross-sectional view of the end cap of FIG. 46;

FIG. 48 illustrates a perspective view of a foot for the tool of FIG. 44;

FIG. 49 illustrates a cross-sectional view of the foot of FIG. 48;

FIG. 50 illustrates a perspective view of a first tibial insertion instrument for the device of FIG. 15;

FIG. 51 illustrates a cross-sectional view of the first tibial insertion instrument of FIG. 50;

FIG. 52 illustrates a perspective view of a second tibial insertion instrument for the device of FIG. 15; and

FIG. 53 illustrates a cross-sectional view of the second tibial insertion instrument of FIG. 52.

### DETAILED DESCRIPTION

The present invention may be further understood with reference to the following description and the appended drawings, wherein like elements are referred to with the same reference numerals. The present invention relates to the treatment of bone joints and, in particular, to a subcutaneously implantable device for distraction of a bone joint. The exemplary system and method according to the invention is described with respect to the distraction of a knee joint. It is noted, however, that the exemplary system and method according to the invention may be used for the treatment of the hip, elbow joints, ankle joints or any other joint in the body without deviating from the scope of the invention. The exemplary device according to the invention comprises first and second bone plates positionable against opposing outer surfaces of a bone joint. The first bone plate comprises a first plate hole and a first longitudinal recess extending therethrough, the first longitudinal recess being open to a side wall of the first bone plate. The second bone plate comprises a second plate hole and a second longitudinal recess extending therethrough, the second longitudinal recess being open to a side wall of the first bone plate. Each of the first and second bone plates comprises one or more contours formed and positioned to ensure conformity of the first and second bone plates to the contour of the bones which they contact in an operative configuration, as will be described in greater detail later on. The device further comprises first and second elongated rods which extend through ends of the two bones forming the bone joint. In another embodiment, the first and second rods may be replaces with screw-like elements which extend only partially into the bone from opposing sides thereof, as will be described in greater detail later on. In an operative configuration, guide pins are inserted through target locations of, for example, a femur and tibia. The first and second rods are then inserted along the pins and into the bones. The first and second bone plates are then inserted over ends of the rods on opposing ends of the bone joint such that the first rod is received within the first and second plate holes and the second rod is inserted into the first and second longitudinal recesses. A first pair of locking screws are then inserted into the first and second plate holes to lock a position of the first rod relative thereto. The second rod is provisionally secured to the first and second bone plates by loosely inserting a washer and screw into the second rod via the first and second longitudinal recesses. A distractor instrument according to the invention is then attached to the first and second bone plates. The distractor instrument engages the device in a manner that the first and second rods may be manipulated to a desired angular and spatial orientation relative to one another. This angular and spatial orientation is predetermined and selected to reduce stresses on the bone joint to permit regeneration of cartilage tissue while still permitting a limited movement of the bones. Once the first and second rods and therefore, the femur and tibia, have been moved to the corrected position relative to one another, the screws connected to the second rod are tightened to lock the position of the device. The distractor instrument is then removed. It should be noted that the terms “proximal” and “distal” as used herein, refer to a direction toward (proximal) and away from (distal) a user of the device. It should also be noted that the terms “medial” and “lateral” as used herein indicate a direction toward (medial) and away from (lateral) a midline of the body of a patient within which the bone fixation device is to be implanted.

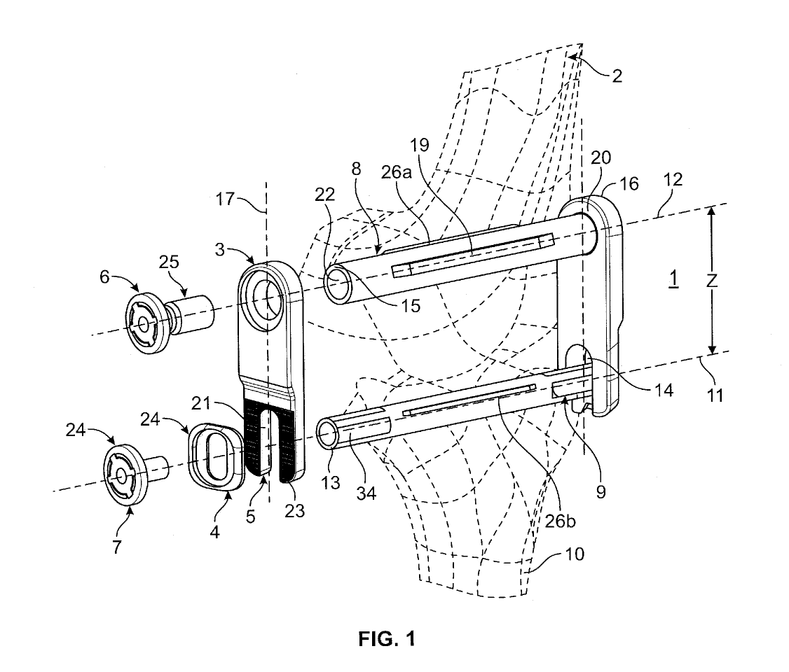

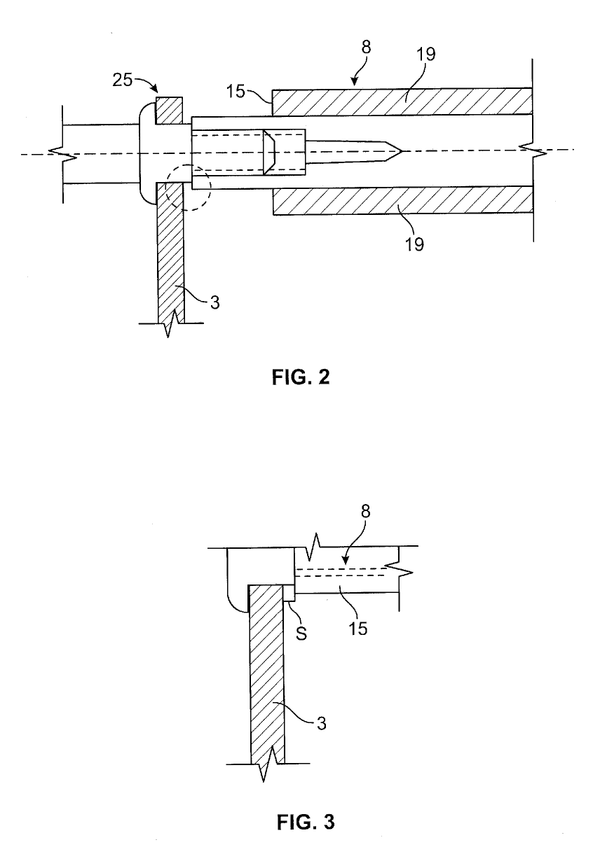

The embodiment of the knee distractor according to FIGS. 1-3 is a subcutaneous hinge based distraction device 1 for the knee joint with the goal to distract and unload the knee joint. It has been shown, that an unloading period for two to three months can be a solution used in the treatment of osteoarthritis. The advantage of the knee distracting device according to the invention is the ability, to flex the knee joint for a certain range of motion by keeping it unloaded, compared to other rigid systems which have to be disassembled every two weeks to allow a certain movement of the joint to keep mobility. The knee distraction device 1 according to the invention is designed to be used subcutaneous which has the advantage to be patient-friendly in terms of daily living and in addition there is no pin tract infection.

The articulations 25 in the form of hinge joints are located as close as possible to the natural center of rotation in the femur 2, whereas on the tibia side, the device 1 consists of an angularly stable rigid fixation. A range of motion of around 30° is sufficient enough to maintain the mobility of the bone joint during the period of distraction. To minimize the risk of wear particles, the hinge joint has an optimized rotation surface which can be treated with an adequate technology to obtain a low friction coefficient.

The device 1 is cannulated on the femur and tibia side for the insertion of the pins 35 which are needed for the distraction instrument. After having distracted the knee distractor device 1 with the help of a forceps like device it can be blocked in the desired position.

The embodiment of the device 1 illustrated in FIGS. 1 to 3 can be intra-operatively assembled by using a kit according to the invention and comprises a first rod shaped member 9 affixed to the tibia 10, a second rod shaped member 8 affixed to the femur 2 and a first and second connecting member 3;20 connecting the first and second rod shaped members 9;8 in such a way that the second rod shaped member 8 is rotatably coupled to the first and second connecting members 3;20 and the first rod shaped member 9 is fixedly coupled to the first and second connecting members 3;20.

The first rod shaped member 9 has a longitudinal axis 11, a first end 13 and a second end 14 and the second rod shaped member 8 has a first end 15, a second end 16 and a longitudinal axis 12 which in the assembled state of the device 1 extends essentially parallel to the longitudinal axis 11 of the first rod shaped member 9. The first and second rod shaped member 9;8 are arranged with a spacing Z>0 between their longitudinal axes 11;12.

Further, the first and second rod shaped member 9;8 each comprise a peripheral surface including anti-rotation means 26a;26b so that the first and second rod shaped members 9;8 are rigidly secured in the femur 2 and the tibia 10 with respect to rotation about their longitudinal axes 11;12. The anti-rotation means 26 each can comprise one or more ridges 19, preferably in the form of a rib or fin extending essentially parallel to the longitudinal axes 11;12 of the first and second rod shaped member 9;8. Additionally, the first and second rod shaped member 9;8 comprise a cannulation 22 for receiving a pin 35 each to which a distractor instrument can be coupled. The anti-rotation means 26a;26b may be positioned on their respective rod-shaped members 9;8 to indicate an alignment of the rod-shaped members.

The first rod shaped member 9 comprises a peripheral surface which is provided with four flattenings 34 each at the first and second end 13;14 so that the first and second ends 13;14 are configured with a generally planar cross section orthogonal to the longitudinal axis 11. The flattenings 34 may indicate an alignment direction of the first rod shaped member 9.

The flattenings 34 and the anti-rotation means 26b, may in combination serve to show an alignment direction of the rod-shaped member 9 relative to the knee joint and also the rod-shaped member 8. For example, the flattenings 34 may indicate where a longitudinal axis 17 of a connecting member 3;20 will be located once in position, the anti-rotation means 26b may indicate where a longitudinal axis 17 of a connecting member 3;20 will be located once in position or both the flattenings and the anti-rotation means 26b may indicate where a longitudinal axis 17 of a connecting member 3;20 will be located once in position. The alignment direction may be chosen by, for example, a surgeon to ensure that when the knee distraction device 1 is installed, the joint members of the knee joint will be in a desired anatomical position.

The first connecting member 3 is plate shaped and can be connected to the first ends 13;15 of the first and second rod shaped member 9;8 and the second connecting member 20 is plate shaped as well and can be connected to the second ends 14;16 of the first and second rod shaped member 9;8. Thereby, the first and second ends 15;16 of the second rod shaped member 8 are joinable to the two connecting members 3;20 by means of an articulation 25, so that the two connecting members 3;20 are rotatable about the longitudinal axis 12 of the second rod shaped member 8.

The articulations 25 are configured as hinge joints each comprising a hinge pin 6 including a cylindrical portion 28 pivotably received in a bore 27 in each one of the first and second connecting members 3;20. Adjacent this cylindrical portion 28 a head 29 with a diameter larger than the diameter of the cylindrical portion 28 is arranged and adjacent the other end of the cylindrical portion 28 a threaded portion 30 with a diameter smaller than the diameter of the cylindrical portion 28 is arranged. The threaded portion 30 of each hinge pin 6 can be screwed in a corresponding internal thread in each end 15;16 of the second rod shaped member 8. At the transition between the head 29 and the cylindrical portion 28 of each hinge pin 6 a first shoulder is formed which rests on one of the first and second connecting members 3;20. At the transition between the cylindrical portion 28 and the threaded portion 30 a second shoulder is formed whereof each second shoulder rests on one of the ends 15;16 of the second rod shaped member 8. Thus, the first and second connecting member 3;20 are each retained between a first shoulder and an end 15;16 of the second rod shaped member 8. The cylindrical portion 28 is slightly longer than the thickness of the first and second connecting members 3;20 so that the first and second connecting members 3;20 are mounted on the hinge pin 6 with an axial clearance S in the direction of the longitudinal axis 12 of the second rod shaped member 8. The sliding surfaces of each articulation 25 are formed by the peripheral surface of the cylindrical portion 28 of the hinge pin 6 and the surface of the bore 27 in each of the first and second connecting member 3;20. These sliding surfaces can be provided with a CrCo, TiN or ADLC (amorphous diamond-like carbon) coating.

The first and second connecting members 3;20 are each reversibly fixable to one of the two ends 13;14 of the first rod shaped member 9, wherein the first and second connecting member 3;20 are each rotatively form-fittingly fixable to one of the two ends 13;14 of the first rod shaped member 9. Due to the rotatively form-fitting fixation of the first and second connecting members 3;20 at the ends 13;14 of the first rod shaped member 9 a rigid support for the second rod shaped member 8 is established. Furthermore, the device 1 comprises reversibly securable adjusting means 5 so that the spacing Z between the longitudinal axes 11;12 of the first and second rod shaped member 9;8 is adjustable and reversibly fixable by means of the adjusting means 5. The sliding surfaces of the knee joint can thus be relieved of the load applied onto the knee joint. The first and second connecting member 3;20 each comprise a longitudinal axis 17;18 and the adjusting means 5 comprise a longitudinal recess 21 extending in the direction of the longitudinal axis 17;18 of the first and second connecting member 3;20. The first rod shaped member 9 comprises a peripheral surface which is provided with flattenings 34 at the first and second end 13;14 which engage with the longitudinal recess 21. Thereto, the width of the longitudinal recess 21 is adapted to engage with two opposite flattenings 34 arranged on the peripheral surface at each end 13;14 of the first rod shaped member 9. Further, the adjusting means 5 each comprise a reversibly fixable securing means 24 including a screw 7 and a washer 4 for securing the first and second connecting member 3;20 each to one end 13;14 of the first rod shaped member 9. Each one screw 7 penetrates through the washer 4 and through the longitudinal recess 21 and can screwed into one end 13;14 of the first rod shaped member 9. In the area of longitudinal recesses 21 the first and second connecting member 3;20 comprise a toothing 23 on their outer surface which engage with a corresponding toothing on the contacting surface of the washer 4. The adjusting means 5 permit to distract the device 1 with pre-mounted but untightened securing means 24 and permit to retain the distracted device 1 in a desired distracted position when the securing means 24 are tightened.

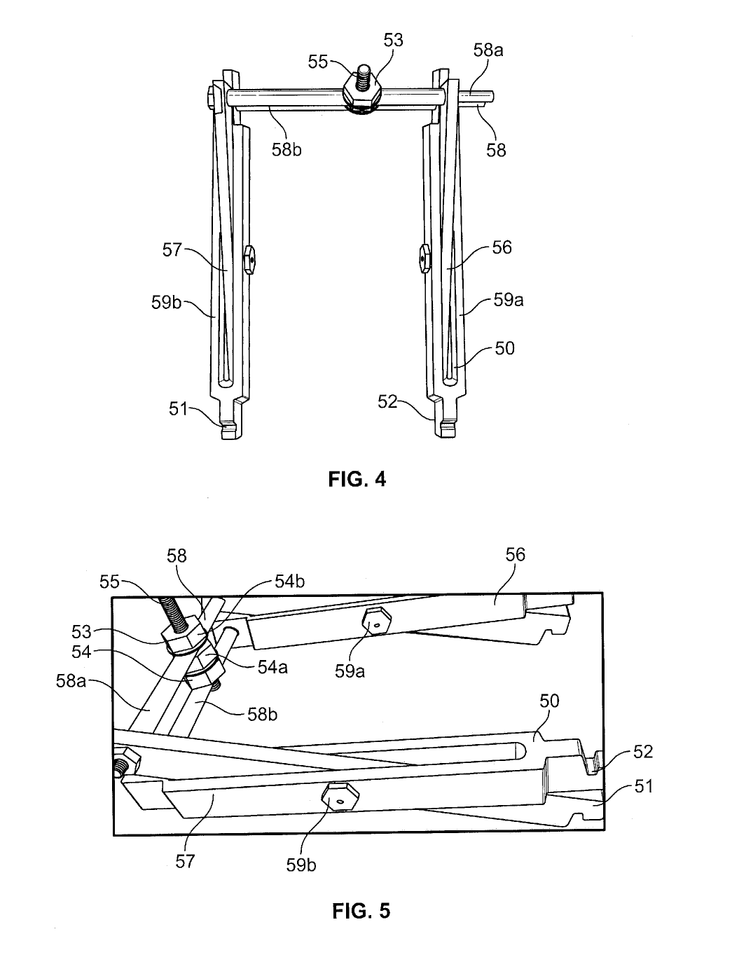

FIGS. 4 to 7 show an instrument 50 for setting a displacement between a first and a second rod-like member 8;9. The instrument 50 has a first holder 51 adapted to hold the rod-like member 9, a second implant holder 52 adapted to hold the rod-like member 8, and a spacing arrangement 53. The spacing arrangement 53 has a displacer 54 arranged to vary a displacement between the first holder 51 and the second holder 52 to set a desired displacement. The spacing arrangement 53 may have also a spacer 55 arranged to hold the first holder 51 spaced apart from the second holder 52 at the desired displacement. The instrument 50 may be a distraction setting instrument for setting a desired distraction displacement between the first and second holders 51, 52.

The first holder 51 is pivotally connected to the second implant holder 52 in a scissor-like arrangement. In the scissor arrangement, first ends of the first and second holders 51;52 are adapted to hold the first and second rod-like members 8;9, respectively. The spacing arrangement 53 is used to space apart and holding the second ends of the first and second implant holders, whilst the first and second rods 8;9 are fixed to the connecting members 3;20 to set the distraction between the members of the knee joint.

The scissor-like arrangement is provided by a first and a second scissor-like member 56;57. The first scissor-like member 56 may be spaced apart from the second scissor-like member 57 by an axes 58 to which the first and second scissor-like members 56;57 are pivotally connected. Opposed second ends of the first and second scissor-like members 56;57 of the first holder 51 may be rotatably connected to a first axis 58a and opposed second ends of the first and second scissor-like members 56;57 of the second holder 52 may be rotatably connected to a second axis 58b. The scissor characteristic is provided by the axes 59a;59b that each located substantially centrally relative to the first and second ends of the scissor-like members 56;57.

The spacing arrangement 53 is connected between the first and second axes 58a;58b. The displacer 54 and spacer 55 are provided by a shaft in combination with a bolt-like element. The shaft is elongate and has an external thread. The bolt-like element has an internal thread that is positioned around the shaft. The bolt-like element is rotatable on the shaft to engage the first holder 51 to vary the displacement between the first and second implant holders 51;52. The shaft has a first end that is rotatably coupled to the second axis 58b. The rotatable coupling is used to allow limited rotation of the shaft that occurs due to the bolt-like element engaging with the first axis to vary the displacement. The other end of the shaft is positioned through a channel in the first axis 58a. The bolt-like element has a first displacement member 54a and a second displacement member 54b. The first displacement member 54a is positioned on the shaft relative to a first opening into the channel in the second displacement member 54 is positioned on the shaft relative an opposed second opening. The first displacement member 54a is arranged to be rotated and increase the distance between the first and second axes 58a;58b to, for example, set a distraction distance. The second displacement member 54b is rotated to reduce the displacement between the first and second axes 58a;58b to, for example, reduce the distraction distance or to remove the instrument 50.

To implant the knee distraction device, firstly, an axis of rotation of the knee joint is determined. This determination may be achieved using a translucent template. Afterwards, a first Kirschner-wire is inserted into the femur 2 by means of a universal drilling machine along the axis of rotation. Then the position of the Kirschner-wire is checked via a C-arm X-ray device and a x-ray translucent template with radio-opaque markers arranged in circles. The axis of the Kirschner-wire has to be positioned through the instantaneous axis of rotation of knee joint and has to extend essentially parallel to the tibia-plateau.

Having positioned the first Kirschner-wire in the femur 2 an adjustable drill guide is adjusted to the correct position, e.g. the correct spacing Z for the second Kirschner-wire in the tibia 10. The drill guide guarantees that the axis of the second Kirschner-wire in the tibia 10 extends parallel to the axis of the first Kirschner-wire in the femur 2. Then, the second Kirschner-wire is positioned in the tibia 10 by means of the drilling machine.

Having positioned the first and second Kirschner-wire the drill guide is removed. The first and second Kirschner-wire can then be used to guide a cannulated reamer or drill, wherein firstly, the bore hole 32 in the femur 2 is drilled by using a reamer with a diameter of 9.5 mm and secondly, the bore hole 31 in the tibia 10 is drilled by using a reamer with a diameter of 7.5 mm. The diameters of the bore holes 32;31 in the femur 2 and in the tibia 10 are selected in such a way that the first rod shaped member 9 with a diameter of 10 mm for the femur 2 and the second rod shaped member 8 with a diameter of 8 mm for the tibia 10 can be beaten in with little force.

Once the two bore holes 32;31 in the femur 2 and in the tibia 10 are reamed the first and second rod shaped members 9;8 can be beaten in each into one of the two bore holes 32;31 in the femur 2 and in the tibia 10. Thereby, the first rod shaped member 9 has to be rotatively aligned so that two parallel flattenings 34 extend essentially parallel to a line connecting the longitudinal axes 11;12 of the first and second rod shaped members 9;8.

Then, each a pin 35 with a diameter of 4 mm is inserted through the cannulations 22 in the first and second rod shaped members 9;8 so that a distractor instrument can be attached to the pins 35 once the device 1 is pre-assembled.

After insertion of the pins 35 the device 1 is pre-assembled by firstly attaching the first and second connecting member 3;20 to the second rod shaped member 8, which is inserted in the femur 2. Thereby, the hinge pins 6 are passed through the bores 27 in the first and second connecting members 3;20 and screwed into the second rod shaped members 8 as far as the second shoulders formed between the cylindrical portions 28 and the threaded portions 30 of the hinge pins 6 abut against the ends 15;16 of the second rod shaped member 8. Secondly, the securing means 24 is pre-mounted by passing a screw 7 through the washer 4 and the recess 21 in each of the first and second connecting member 3;20 and loosely screwing the screw 7 in each of the corresponding interior threads at the first and second end 13;14 of the first rod shaped member 9.

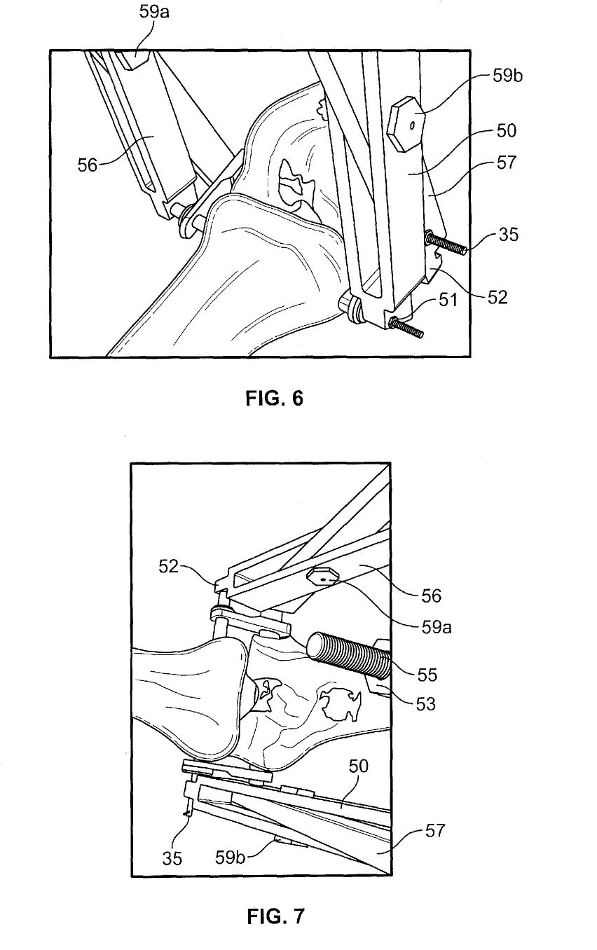

The device 1 is now completely assembled. As shown by FIGS. 6 and 7 the distractor instrument 50 is then attached to the pins 35 and the first and second rod shaped members 9;8 and consequently the femur 2 and the tibia 10 are distracted step-wise to about 5 mm by using the distractor instrument 50. Once the first and second rod shaped member 9;8 have been distracted to the desired spacing Z the screws 7 are tightened to therewith rigidly fix the first and second connecting member 3;20 to the first rod shaped member 9.

Finally, the distractor instrument and the pins 35 can be removed from the device 1 and the incisions can be closed.

For the implantation of the device 1 each a lateral and a medial puncture incision in the area of the knee joint can be performed to permit a minimal invasive implantation of the device 1. Alternatively, each an incision traversing the knee joint can be performed to implant the device 1.

After healing of the knee joint over a period of 2 to 3 months the device 1 can be explanted.

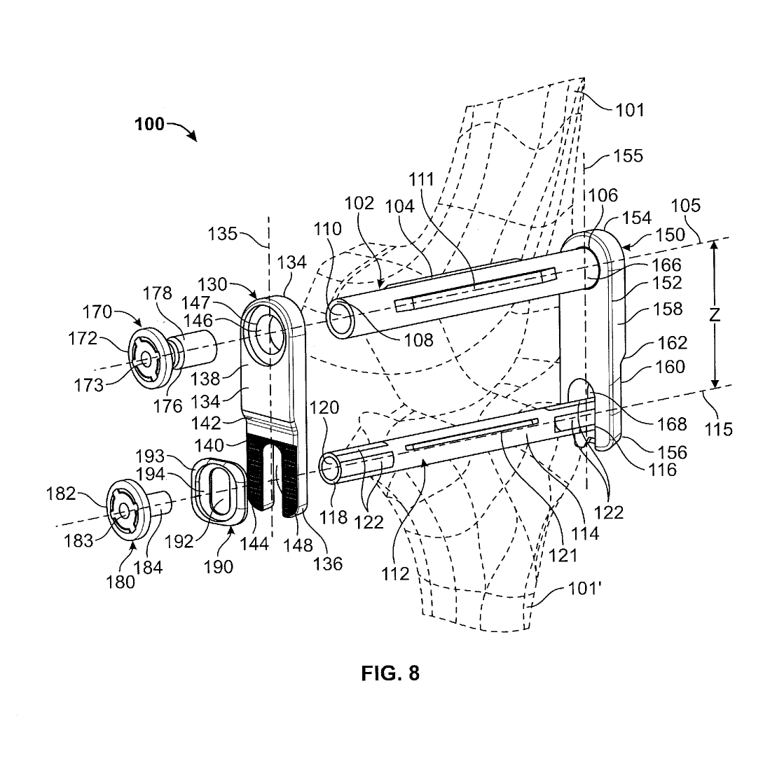

FIGS. 8-10 depict an exemplary subcutaneous hinge based knee distraction device 100 according to the invention. The knee distraction device 100 is formed substantially similarly to the device 1 of FIGS. 1-7, the following description being provided as a further detailed description of the device 1. The exemplary device 100 is used for distraction of the knee joint with the goal to distract and unload stresses applied thereto. It has been shown, that an unloading period for two to three months can be a solution used in the treatment of osteoarthritis. It is noted, however, that the device 100 is implantable for any period of time without deviating from the scope of the invention. The exemplary device 100 provides the ability to flex the knee joint through a certain range of motion while keeping it unloaded. In contrast, other rigid systems available in the art must be disassembled every two weeks to allow certain movement of the joint to retain mobility. The device 100 according to the invention is designed to be used subcutaneously, which has the advantage of being patient-friendly in terms of daily living and preventing pin tract infection.

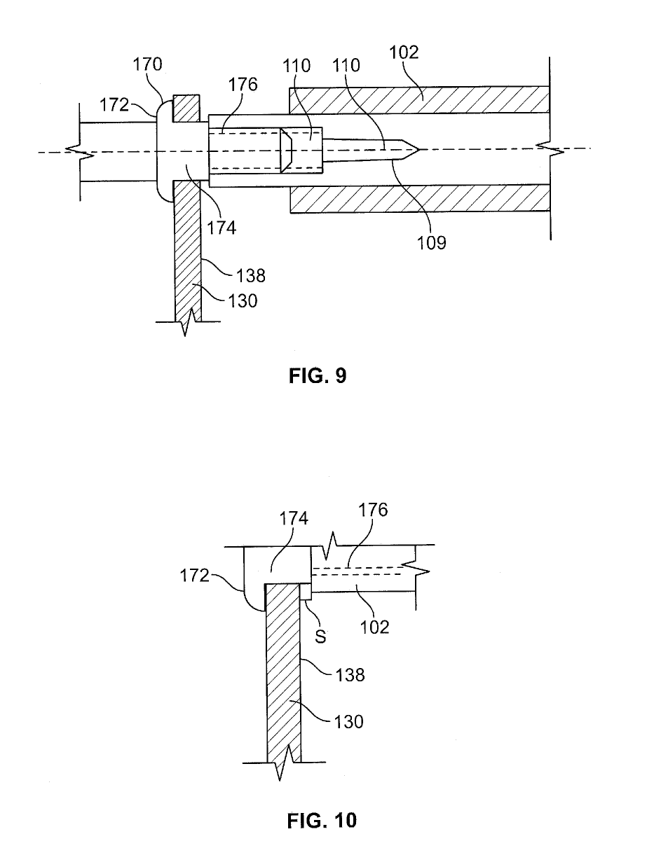

The device 100 comprises a first elongated rod 102 inserted into a femur 101 and a second elongated rod 112 inserted into a tibia 101′. The first elongated rod 102 includes a shaft 104 extending along a longitudinal axis 105 from a medial end 106 to a lateral end 108 and includes openings 110 formed at both ends 106, 108. Specifically, as shown in greater detail in FIG. 9, the opening 110 extends into the first rod 102 from the lateral end 108 for a predetermined length conforming to a length of a first hinge pin 170 to be received therein, as will be described in greater detail later on. The opening 110 may be threaded to correspond to a threading of the first hinge pin 170. The opening 110 may further comprise a recess portion 109 at an end thereof and extending into the first rod 102 for a predetermined distance. A diameter of the recess portion 109 in this embodiment is smaller than that of the opening 110 and terminates in a pointed tip. As those skilled in the art will understand, the recess portion 109 is provided to absorb stresses applied to the opening 110 and the first rod 102 during and after implantation. The shaft 104 may include one or more ridges 111 distributed over an outer surface thereof to prevent rotation of the first rod 102 about the longitudinal axis 105 when positioned within the femur 10. The ridges 111 are formed as projections extending radially out of the shaft 104 a distance selected to increase frictional resistance and purchase within the femur 101 to prevent and/or limit rotation of the first rod 102 relative to the femur. The ridges 111 may, for example, extend substantially parallel to a longitudinal axis of the first rod 102 and may be formed with a length smaller than a length of the first rod 102. In a first exemplary embodiment, the shaft 104 includes three ridges 111 evenly distributed over a circumference thereof, although any other number and location of ridges may be used without deviating from the scope of the invention. The device 100 further comprises a second elongated rod 112 including a shaft 114 extending along a longitudinal axis 115 from a medial end 116 to a lateral end 118 with a channel 120 extending therethrough. In another embodiment, the channel 120 may be formed as openings extending into the medial and lateral ends 116, 118 over a predetermined distance selected to accommodate insertion of a second hinge pin 180 thereinto, as will be described in greater detail later on. The shaft 114 may include one or more ribs 121 or fins distributed over a circumference thereof to prevent rotation of the second rod 102 about the longitudinal axis 115 when positioned within the tibia 101′ in the same manner described above in regard to the ridges 111. The ribs 121 are formed as projections extending radially out of the shaft 114 a distance selected to increase frictional resistance and purchase in the tibia 101′ thus preventing and/or limiting rotation of the second rod 112 therewithin. The ribs 121 may extend substantially parallel to a longitudinal axis of the second rod 112 and may be formed with a length smaller than a length of the second rod 112. In a first exemplary embodiment, the shaft 114 includes three ribs 121 evenly distributed over the circumference thereof, although any other number and distribution of ribs may be used without deviating from the scope of the invention.

The shaft 114 further comprises flattenings 122 adjacent medial and lateral ends 116, 118 thereof. In an exemplary embodiment, each of the medial and lateral ends 116, 118 includes four flattenings 122 on an outer surface thereof such that the medial and lateral ends 116, 118 have a substantially planar cross section orthogonal to the longitudinal axis 115. The flattenings 122 indicate an alignment direction of the second rod 112, as will be described in greater detail later on. The flattenings 122 and the ribs 121 may, in combination, serve to show a desired alignment direction of the second rod 112 relative to both the knee joint and the first rod 102. For example, the flattenings 122 and ribs 122, either alone or in combination, may indicate where a longitudinal axis 135,155 of one of the first and second plates 130, 150 will be located once in position. Similarly, the ridges 111 may be used to indicate an alignment of the first rod 102 in an operative configuration. The alignment direction may be chosen by, for example, a surgeon to ensure that when the device 100 is implanted, the joint members of the knee joint will be in a desired anatomical position.

The device 100 further comprises the first plate 130 which includes an elongated plate body 132 from a first end 134 to a second end 136 along a longitudinal axis 135. The body 132 includes a first portion 138 having a first thickness and a second portion 140 having a second thickness smaller than the first thickness. The first and second portions 138, 140 are separated from one another by an angled wall 142. A length and thickness of each of the first and second portions 138, 140 as well as an angle of the angled wall 142 is selected to conform to a contour of a bone of the bone joint to be corrected, as those skilled in the art will understand. The second portion 140 further comprises serrations 144 on a lateral surface thereof. The serrations 142 are formed as a plurality of pointed teeth extending laterally away from the second portion 140. As will be described in greater detail below, the serrations 144 are formed to engage corresponding serrations formed on a washer 190 positioned thereagainst in an operative configuration.

The first portion 138 of the first plate 130 further comprises a first plate hole 146 adjacent the first end 134. The first plate hole 146 is substantially cylindrical and has a diameter closely matching a diameter of the first rod 102 to permit insertion of the first plate 130 thereover in an operative configuration, as will be described in greater detail with respect to the exemplary method below. A lateral end of the first plate hole 146 includes a beveled lip 147 to permit seating of a head 172 of a first hinge pin 170 inserted thereinto, as those skilled in the art will understand. The second portion 140 of the first plate 130 comprises a first longitudinal recess 148 extending therethrough and open to the second end 136. The recess 148 extends parallel to the longitudinal axis 135. A width of the longitudinal recess 148 is substantially the same as a width of the second rod 112 at the flattenings 122. Thus, the second rod 112 is insertable into the recess 148 in only those configurations where a pair of the flattenings 122 extend parallel to the longitudinal axis 135.

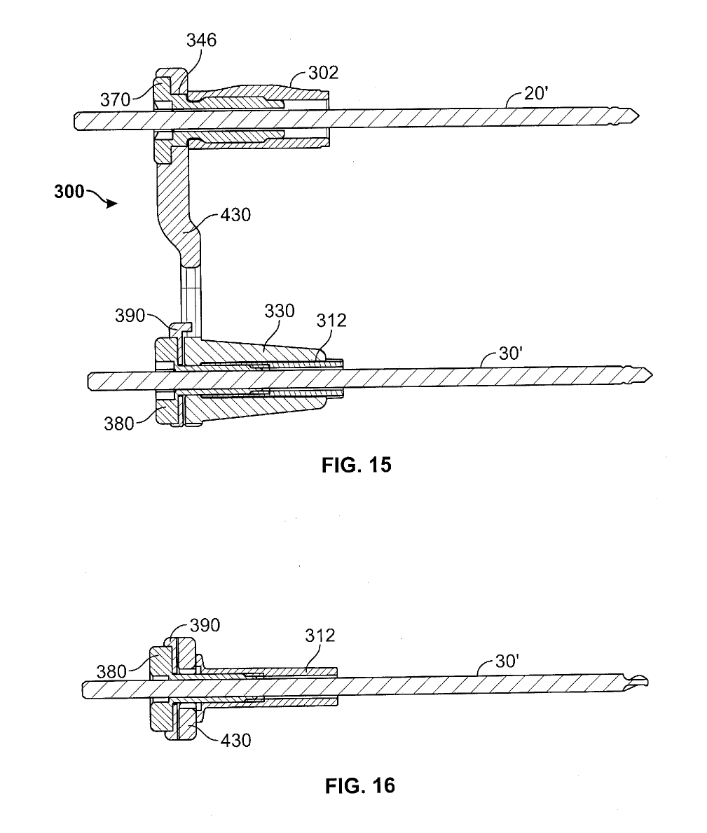

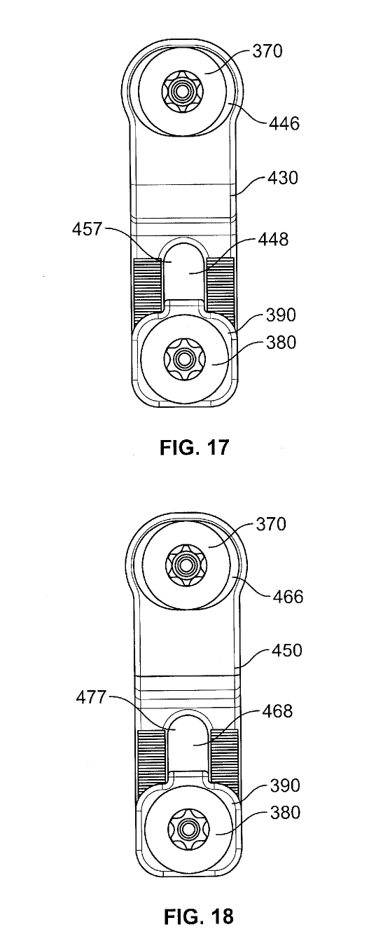

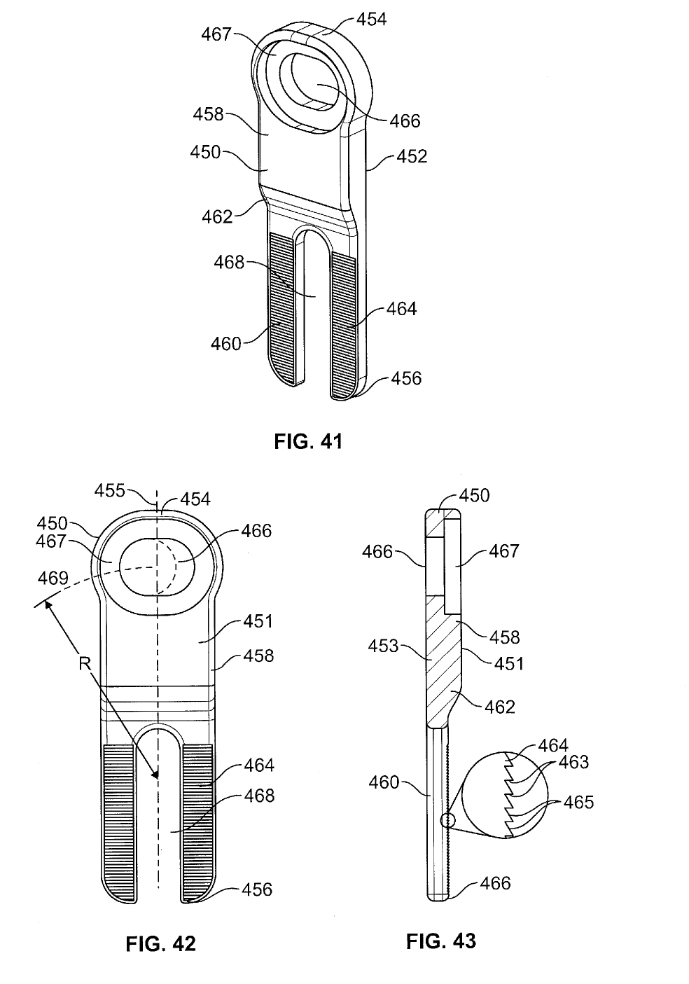

The second plate 150 is formed substantially similarly to the first plate 130. Thus, a surgeon or other user may use the first and second plates 130, 150 interchangeable. In another embodiment, as shown in FIGS. 15-52, first and second plates may be formed with different geometries to permit conformation thereof to particular contours of the lateral and medial ends of a bone joint. The second plate 150 includes an elongated plate body 152 extending from a first end 154 to a second end 156 along a longitudinal axis 155. The body 152 includes a first portion 158 having a first thickness and a second portion 160 having a second thickness smaller than the first thickness, the first and second portions 158, 160 being separated from one another by an angled wall 162. The second portion 160 further comprises serrations (not shown) provided on a medial surface thereof, the serrations (not shown) formed as a plurality of pointed teeth extending medially away from the second portion 160 and substantially similar to the serrations 144, as described in greater detail earlier.

The first portion 158 of the second plate 150 further comprises a second plate hole 166 adjacent the first end 154. The second plate hole 166 is substantially similar to the first plate hole 136 and has a diameter closely matching a diameter of the first rod 102 to permit insertion of the second plate 150 thereover in an operative configuration, as will be described in greater detail with respect to the exemplary method below. A medial end of the second plate hole 166 includes a beveled lip (not shown) formed similar to the beveled lip 147. The second portion 160 of the second plate 150 comprises a second longitudinal recess 168 extending therethrough and open to the second end 156. The second longitudinal recess 168 extends parallel to the longitudinal axis 155 and is formed substantially similarly to the first longitudinal recess 148.

The device 100 further comprises a first hinge pin 170 having a head 172, a first shaft portion 174, and a second shaft portion 176 extending therefrom. The head 172 may include a recess 173 or other mechanism to permit engagement thereof with a driving mechanism, as those skilled in the art will understand. A diameter of the head 172 is greater than the diameter of the first and second plate holes 146, 166 and the openings 110 to prevent the head 172 from being inserted thereinto. In an operative configuration, at least a portion of the head 172 is seated within the beveled lip 147 of the first and second plate holes 146, 166. A diameter of the first shaft portion is substantially the same as a diameter of the first and second plate holes 146, 166 and larger than a diameter of the opening 110. Thus, in an operative configuration, only the second shaft portion 176 is insertable into the opening 110 of the first rod 102, as shown in greater detail in the partial cross-sectional views of FIGS. 9 and 10. Furthermore, a length of the first shaft portion 174 is greater than a thickness of the first portion 138 of the first plate 130. Thus, when the hinge pin 170 is inserted through the first plate hole 146 and into the opening 110, a clearance S is formed between the first rod 102 and the first plate 130, as shown in FIG. 10. As those skilled in the art will understand, this configuration reduces frictional wear between bearing surfaces of the first hinge pin 170 and the first plate 130. Bearing surfaces between the first hinge pin 170 and the second plate 150 may be similarly treated to reduced friction therebetween. The second shaft portion 176 is formed with threading 178 corresponding to threading (not shown) formed on a wall of the opening 110. In an exemplary embodiment, one or both of the second shaft portion 176 and the walls of the opening 110 may be provided with a CrCo, TiN or ADLC (amorphous diamond-like carbon) coating. In an operative configuration, one hinge pin 170 is provided through the first plate hole 146 and into the lateral end 108 of the first rod 102 and a second hinge pin 170 is provided through the second plate hole 166 and into the medial end of the first rod 102. In this configuration, prior to the insertion of any hinge pins into the first and second longitudinal recesses 148, 168, the first and second plates 130, 150 are rotatable about the longitudinal axis 105 of the first rod 102.

The device 100 further comprises a second hinge pin 180 and a washer 190 insertable through the first and second longitudinal recesses 148, 168 and into the second rod 112. The second hinge pin 180 includes an increased diameter head 182 and an elongated shaft 184 extending therefrom. The head 182 includes a recess 183 or other mechanism to permit engagement thereof with a driving mechanism, as those skilled in the art will understand. The washer 190 includes a washer opening 192 extending therethrough. A lateral surface 193 of the washer 190 bordering the washer opening 192 includes a beveled lip 194 formed to house at least a portion of the head 182 therein. A medial surface (not shown) of the washer 190 includes a plurality of serrations (not shown) formed to interlock with the serrations 144 of the first plate 130. A length of the washer 190, taken along the longitudinal axis 135, is smaller than a length of the second portion 140 of the plate 102, thus permitting a surgeon or other user to selectively position the washer 190 therealong to conform to the requirements of a procedure. A diameter of the washer opening 192 is substantially equivalent to a width of the first and second longitudinal recesses 148, 168 and is selected to permit insertion of the shaft 184 therethrough and into the second rod 112. The shaft 184 and outer wall of the opening 118 may be threaded to permit threaded engagement of the second hinge pin 180 with the second rod 112, as will be described in greater detail with respect to the exemplary method below.

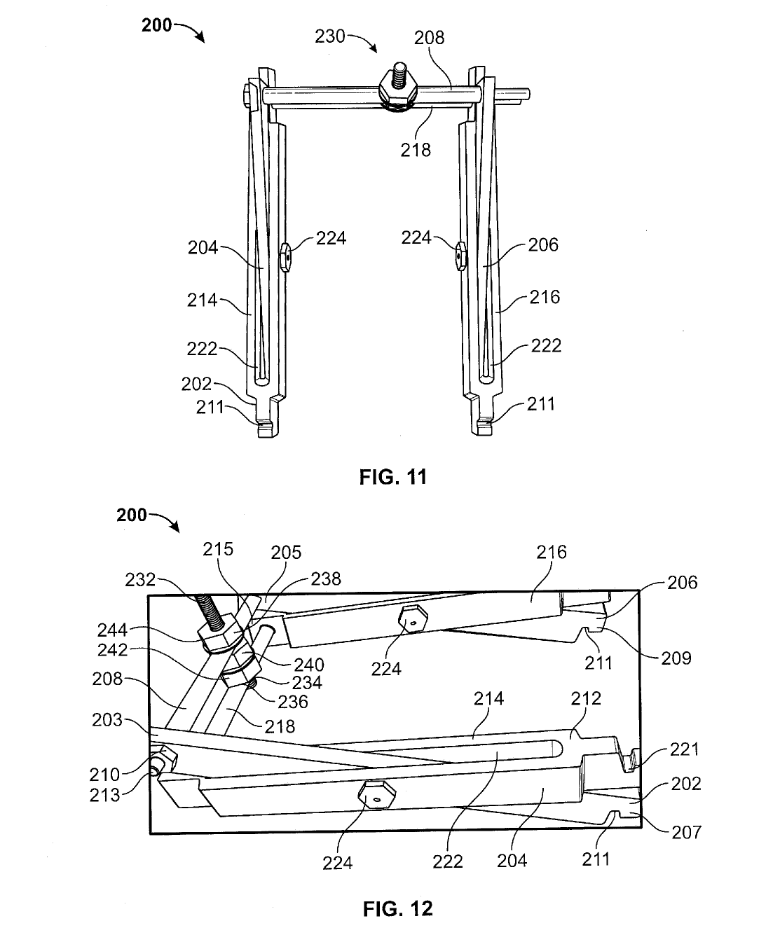

FIGS. 11-12 depict a distraction instrument 200 according to a first exemplary embodiment of the invention. In an operative configuration, the distraction instrument 200 is attachable to the device 100 to displace the first and second rods 102, 112 to a desired spatial relationship relative to one another, as will be described in greater detail later on. The instrument 200 includes a first holding arrangement 202 adapted to hold the first rod 102 and a second holding arrangement 212 adapted to hold the second rod 112. The first holding arrangement 202 includes first and second arms 204, 206 separated from one another by a first spacer 208. In an exemplary embodiment, the first spacer 208 may be received through openings formed in first ends 203, 205 of the first and second arms 204, 206 and secured thereto by a locking screw or nut 210. Second ends 207, 209 of the first and second arms 204, 206 include hooks 211 formed to receive the first rod 102 therein. A width of each of the first and second arms 204, 206 of the first holding arrangement 202 is selected to permit slidable insertion thereof into the second holding arrangement, as described in greater detail below.

The second holding arrangement 212 includes first and second arms 214, 216 separated from one another by a second spacer 218. In an exemplary embodiment, the second spacer 218 may be received through openings formed in first ends 213, 215 of the first and second arms 214, 216 and secured thereto by a locking screw. Second ends 217, 219 of the first and second arms 214, 216 include hooks 221 formed to receive the second rod 112 therein. Each of the first and second arms 214, 216 includes a longitudinal slot 222 extending therethrough to receive the first and second arms 204, 206, respectively, therethrough. The first and second holding arrangements 202, 212 may be pivotally connected to one another by first and second pivot pins 224 to form a scissor-like arrangement. In an exemplary embodiment, the pivot pins 224 are located along a mid-point of the first and second arms 204, 214, 206, 216.

The instrument 200 further comprises a spacing arrangement 230 connected between the first and second spacers 208, 218. The spacing arrangement 230 comprises an elongated shaft 232 extending from a first end (not shown) to a second end 234 having an opening 236 extending therethrough, the opening 236 being formed to permit slidable insertion of the second spacer 218 therethrough. The shaft 232 includes an external thread which threadedly receives a bolt 238 thereover. The bolt 238 includes an opening 240 extending therethrough to receive the first spacer 208 therethrough. The bolt 238 further comprises first and second displacement members 242, 244 located along the shaft 232 on opposing sides of the bolt 238. The first displacement member 242 is arranged to be rotated about the shaft 232 to increase the distance between the first and second spacers 208, 218 and thereby, a corresponding distance between hooks 211, 221. The second displacement member 244 is rotatable about the shaft 232 to reduce the displacement between the first and second spacers 208, 218 to, for example, reduce a distraction distance or to remove the instrument 200, as will be described in greater detail later on. The first and second displacement members 242, 244 may be formed as hexagonal bolts actuatable manually or by a corresponding rotation instrument (e.g., a wrench, etc.). In an operative configuration, the first and second displacement members 242, 244 may be positioned to adjust a spacing Z between longitudinal axes 105, 115 of the first and second rods 102, 112 connected to the instrument 200.

In accordance with an exemplary method according to the invention, a desired axis of rotation of the knee joint is determined. Specifically, a surgeon or other user determines a desired axis of rotation of the femur relative to the tibia. This determination may be achieved using a translucent template, as those skilled in the art will understand. Medial and lateral puncture incisions are formed in the area of the knee joint to permit a minimally invasive implantation of the device 100. Alternatively, a single incision traversing the knee joint can be performed to implant the device 100. A first Kirschner wire 20′ is then inserted into the femur 101 by means of a universal drilling machine along the axis of rotation of the femur 10. The position of the Kirschner wire 20′ is checked via a C-arm X-ray device and a x-ray translucent template with radio-opaque markers arranged in circles, as those skilled in the art will understand. It is noted, however, that any other position verification mechanism may be used without deviating from the scope of the invention. An axis of the Kirschner wire 20′ is positioned through an instantaneous axis of rotation of knee joint and extends substantially parallel to the tibia-plateau. An adjustable drill guide (not shown) is then adjusted to a position to guide a second Kirschner wire 30′ into the tibia 101′ such that the first and second Kirschner wires 20′, 30′ extend parallel to one another and are separated from one another by a space Z. The second Kirschner wire 30′ is positioned in the tibia 101′ by means of the drilling machine (not shown). The drill guide (not shown) is then removed. The first and second Kirschner wires 20′, 30′ can then be used to guide a cannulated reamer or drill (not shown) therealong and into the femur 101 and tibia 101′, respectively. In an exemplary embodiment, a bore hole formed along the Kirschner wire 20′ into the femur 101 is drilled by using a reamer with a diameter of 9.5 mm to accommodate the first rod 102 having a diameter of 10 mm therethrough. A bore hole formed along the Kirschner wire 30′ in the tibia 101′ is drilled by using a reamer with a diameter of 7.5 mm to accommodate the second rod 112 having a diameter of 8 mm. As those skilled in the art will understand, the difference in diameters of the bore holes and the first and second rods 102, 112 requires that an impacting force be used to insert the first and second rods 102, 112 into the femur 101 and tibia 101′ further increasing frictional engagement between the first and second rods 102, 112 and the surrounding bone to maintain the positions thereof and prevent rotation after implantation. In a next exemplary step, the first and second rods 102, 112 are beaten into the bore holes. During this step, the second rod 112 is rotatively aligned so that two parallel flattenings 122 extend substantially parallel to a line connecting the longitudinal axes 105, 115 of the first and second rods 102, 112 (e.g., a line extending parallel to the axis 135 of the first plate 135 of FIG. 8). In this configuration, ends of the pins 20′, 30′ extend beyond ends of the first and second rods 102, 112 to permit engagement thereof with the distractor instrument 200.

In a next exemplary step, the first and second plates 130, 150 are positioned over the first and second rods 102, 112. First hinge pins 170 are completely inserted through first and second plate holes 146, 166 of the first and second plates 130, 150 and into openings 110 at ends 106, 108 of the first rod 102, as shown in FIGS. 9-10. Second hinge pins 180 are then inserted through washer openings 192 of respective washers 190, through respective first and second longitudinal recesses 148, 168 and into ends 116, 118 of the second rod 112 such that the shaft 184 loosely engages threads of the channel 120.

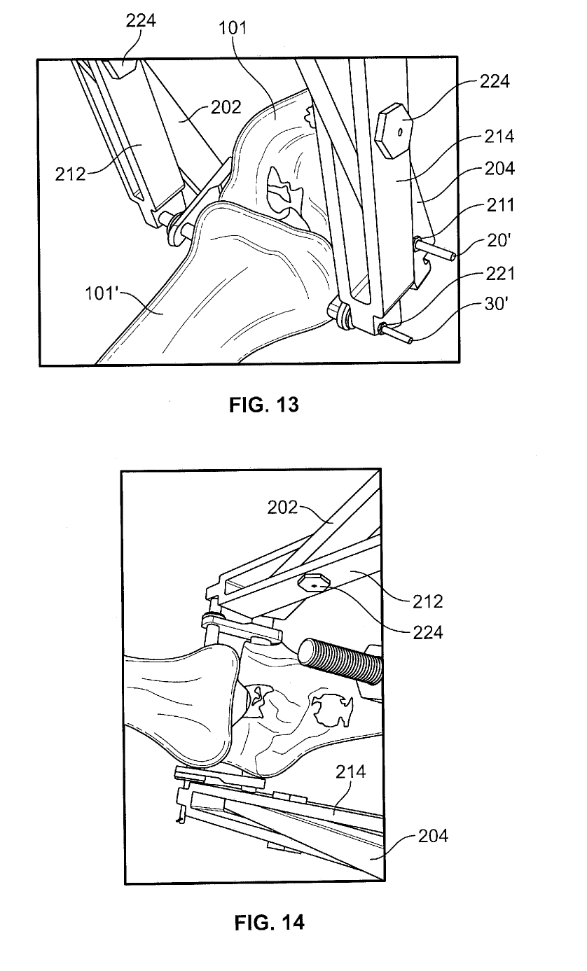

As shown in FIGS. 13-14, the instrument 200 is then attached to the Kirschner wires 20′, 30′ and consequently, to first and second rods 102, 112. Specifically, the Kirschner wires 20′, 30′ are received within the hooks 211, 221. The spacing arrangement 230 is then manipulated to cause a desired distraction of the knee joint. For example, the spacing arrangement 230 may be manipulated to cause a distraction of about 5 mm. Once the first and second rods 102, 112 have been distracted to the desired spacing Z, the second hinge pins 180 are tightened to rigidly engage the first and second plates 130, 150 to the second rod 112. The distractor instrument 200 and Kirschner wires 20′, 30′ are then removed from the device 100 and incisions closed. A permitted range of motion of around 30° is sufficient to maintain the mobility of the bone joint during the period of distraction. To minimize the risk of wear, the device 100 has an optimized rotation surface which can be treated with an adequate technology to obtain a low friction coefficient, as described above with respect to the bearing surfaces of the first hinge pin 170 and the first and second plates 130, 150. In an exemplary embodiment, the device 100 is implanted for approximately two to three months to allow the knee joint to heal. The device 100 is then removed. However, longer or shorter periods of time may be selected by the user based on patient specific considerations.

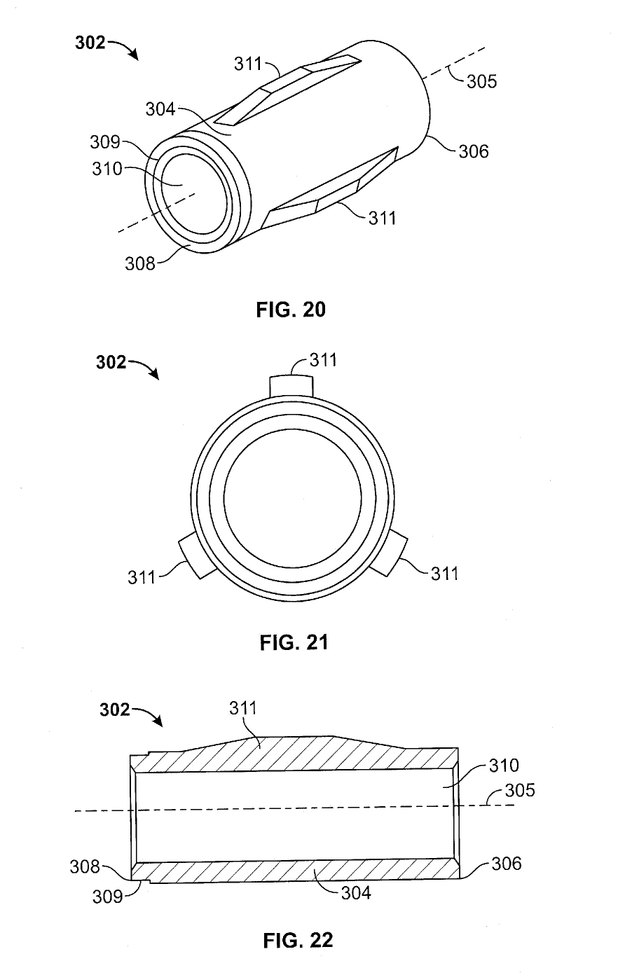

FIGS. 15-53 depict components of a device 300 according to another exemplary embodiment of the invention. The device of FIGS. 15-53 is formed substantially similarly to the device 100 except as noted below. FIGS. 20-22 depict a first rod 302 for insertion into a femur (not shown). In contrast to the device 100 which employs a single elongated rod 102 inserted into the femur, the device 300 employs two substantially shorter first rods 302 inserted into each of medial and lateral portions of the femur, as will be described in greater detail with respect to the exemplary method of this embodiment. Each of the first rods 302 is dimensioned to extend into the femur a predetermined length shorter than a thickness of the bone. As those skilled in the art will understand, this configuration eliminates the requirement to drill a hole through the bone. Rather, a first thin channel is bored therethrough, a diameter thereof being selected to house only a guide pin (e.g., a Kirschner wire) therethrough. A shallow opening is formed at opposing ends of this channel to permit insertion of the first rods 302 thereinto. The first rod 302 includes a shaft 304 extending along a longitudinal axis 305 from a first end 306 to a second end 308 and includes a substantially cylindrical channel 310 extending longitudinally therethrough and open at first and second ends 306, 308. A diameter of the channel 310 is selected to permit insertion of a first hinge pin 370 thereinto, as will be described in greater detail later on. The channel 310 may be threaded to correspond threading of the first hinge pin 370. In another embodiment, the channel 310 and first hinge pin 370 may include no threading, the first hinge pin 370 instead being press-fit into the channel 310. The second end 308 of the first rod 302 further comprises a reduced diameter lip 309 to aid in proper orientation of the first rod 302 into the femur. An outer wall of the shaft 304 may include one or more ridges 311 distributed over an outer surface thereof to prevent rotation of the first rod 302 about the longitudinal axis 305 when positioned within the femur. The ridges 311 are formed as substantially arc-shaped projections extending radially out of the shaft 304 by a distance selected to increase frictional resistance and purchase within the femur thus preventing and/or limiting rotation of the first rod 302 therewithin. The ridges 311 may extend substantially parallel to a longitudinal axis of the first rod 302 and may be formed with a length smaller than that of the first rod 302. In an exemplary embodiment, the shaft 304 may include three ridges 311 evenly distributed over an outer surface thereof, although any other number of ridges 311 may be used without deviating from the scope of the invention.

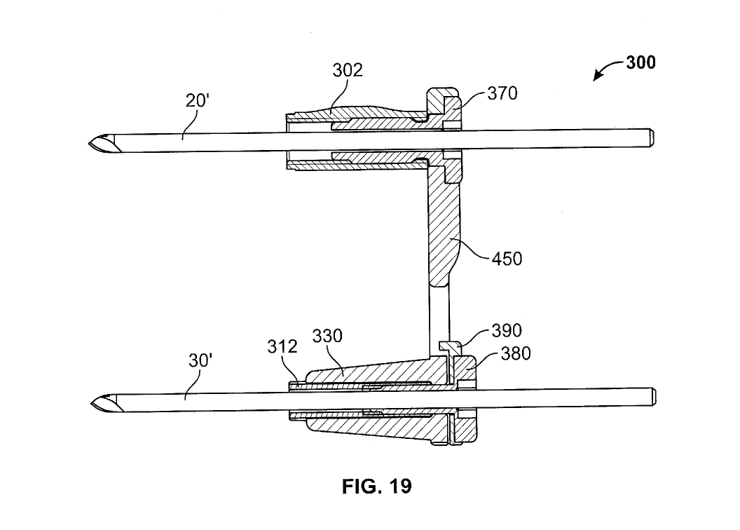

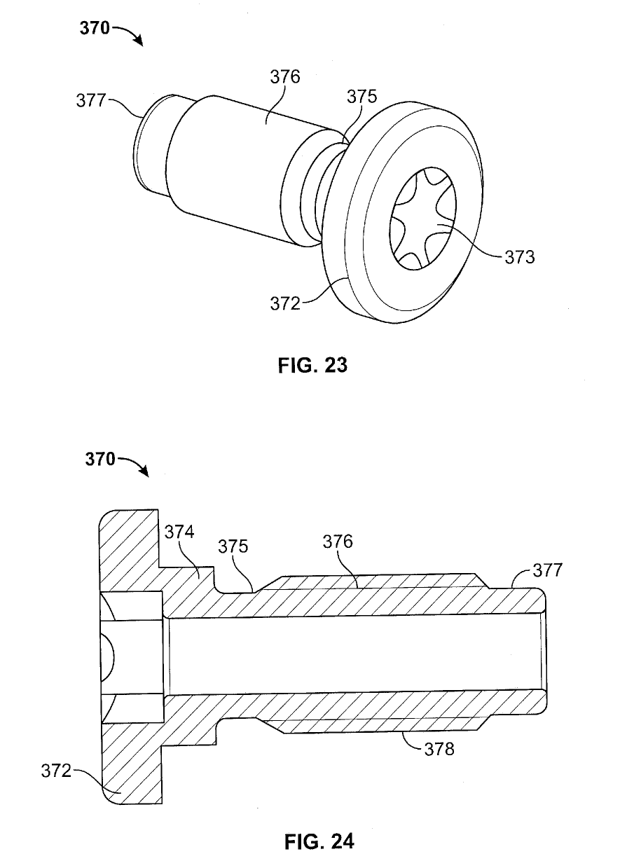

In an operative configuration, the channel 310 engages a first hinge pin 370 formed substantially similarly to the first hinge pin 170. As shown in FIGS. 23-24, the first hinge pin 370 includes a head 372, a first shaft portion 374, and a second shaft portion 376 extending therefrom. The head 372 may include a recess 373 or other mechanism to permit engagement thereof with a driving mechanism, as those skilled in the art will understand. A diameter of the head 372 is greater than the diameter of first and second plate holes 346, 366 and the channel 110 to prevent the head 372 from being inserted thereinto. In an operative configuration, at least a portion of the head 372 is seated within a beveled lip 347 of the first and second plate holes 346, 366. A diameter of the first shaft portion 374 is substantially the same as a diameter of the first and second plate holes 346, 366 and larger than a diameter of the channel 310. Thus, in an operative configuration, only the second shaft portion 376 is insertable into the channel 310 of the first rod 302, as shown in greater detail in FIGS. 15 and 19. Similar to the first hinge pin 170, a length of the first shaft portion 374 may be greater than a thickness of the first portion 338 of the first plate 330. Thus, when the first hinge pin 370 is inserted through the first plate hole 346 and into the channel 310, a clearance S is formed between the first rod 302 and the first plate 330. As those skilled in the art will understand, this configuration reduces frictional wear between the first rod 302 and the first plate 330. In another embodiment, the length of the first shaft portion 374 may be selected to prevent the first shaft portion 374 from extending out of the first plate hole 346. The first hinge pin 370 further comprises a reduced diameter neck 375 between the first and second shaft portions 374, 376 and a reduced diameter distal tip 377 at a free end thereof.

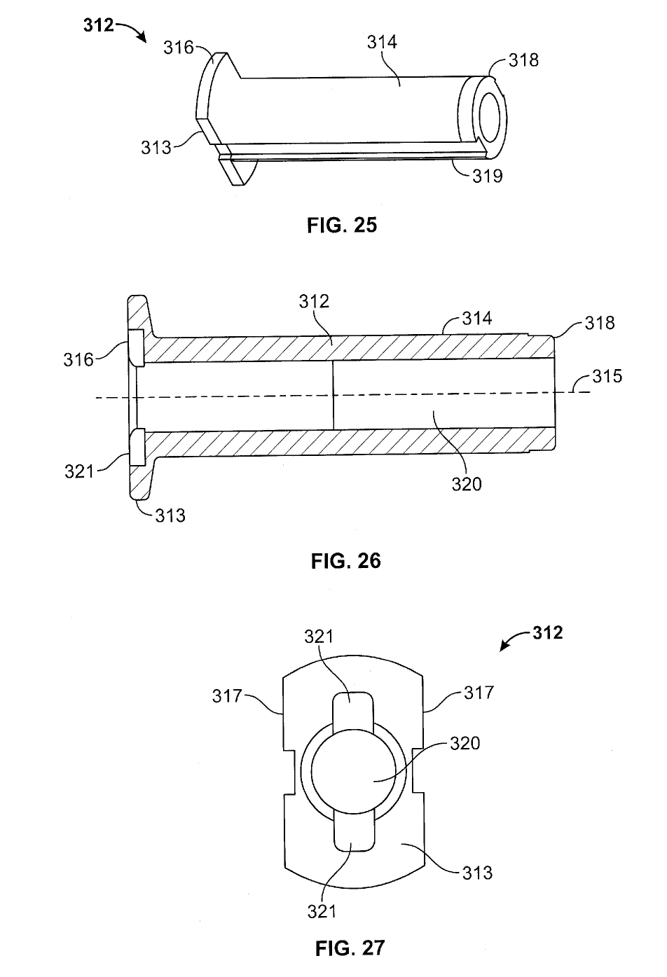

The device 300 further comprises a second elongated rod 312 for insertion into the tibia. Whereas the second rod 112 of the device 100 is dimensioned to be open to both medial and lateral surfaces of the bone in an operative configuration, the second rod 312 extends into medial and lateral ends of the bone by only a predetermined depth, thus eliminating the need to drill a large diameter bore through the bone. As shown in greater detail in FIGS. 25-27, the second rod 312 extends from a first end 316 having an increased diameter head 313 and along a shaft 314 to a second end 318. The head 313 further comprises a non-circular recess 321 formed to engage a driving mechanism, as will be described in greater detail later on. The second rod 312 extends along a longitudinal axis 315 and includes a channel 320 extending therethrough. A cross-sectional shape of the increased diameter head 313 is formed as a circle having flattenings 317 on opposing lateral sides thereof. The flattenings 317 reduce an overall diameter of the head 313 such that the second rod 312 can be inserted through one of the first and second recesses 448, 468, as will be described in greater detail later on. The flattenings 317 are aligned with corresponding longitudinal slots 319 extending along a length of the second rod, as shown in FIG. 25. Dimensions of the slots 319 are selected to permit slidable engagement thereof with arms 336, 340 of a blade 330, as described in greater detail hereinafter. In another embodiment, the second rod 312 may be inserted into the bone prior to positioning of the first or second plates 430, 450 thereover. In this embodiment, the flattenings 317 may be omitted such that a diameter of the second rod 312 is greater than the width of the first and second recesses 448, 468. This increased diameter further aids in maintaining a position of the second rod 312 within the bone, thus eliminating the need to rotate the second rod 312 after insertion thereof into the bone, as will be discussed in greater detail with respect to the exemplary method below.

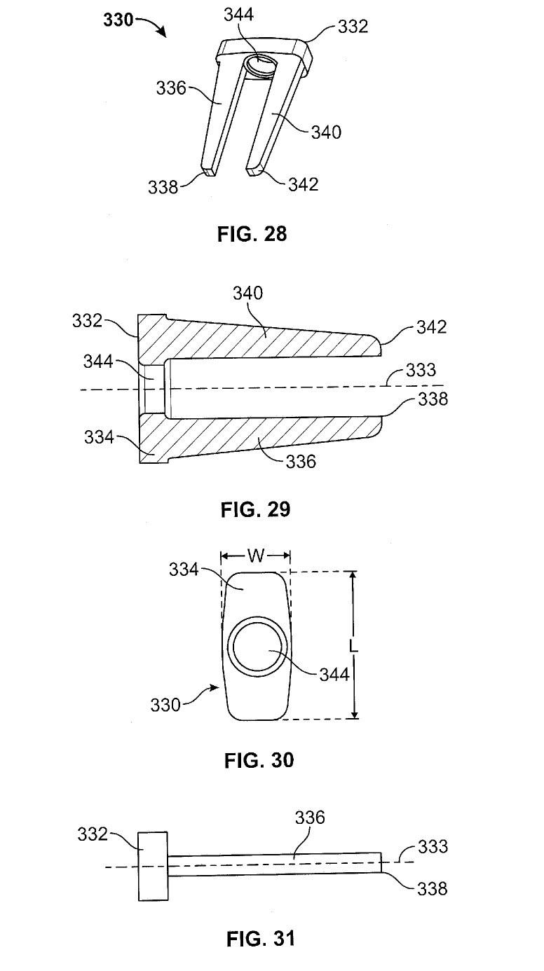

As shown in FIGS. 28-31, the blade 330 extends from a proximal end 332 having a head 334 and includes a pair of arms 336, 340 extending to respective distal ends 338, 342. The head 334 has an elongated substantially rectangular cross-section shaped to be received within one of the first and second recesses 448, 468. Specifically, a width W of the head 334 is substantially equivalent to a width of the recess 448, 468 such that the head 334 is seated therewithin in an operative configuration. A width of the arms 336, 340, measured along an axis extending transverse to a longitudinal axis 333, tapers in a distal direction to terminate at rounded distal ends 338, 342. A length L of the head 334 is greater than the width of the first and second recesses 448, 468. An opening 344 extends through the head 332 and is dimensioned to correspond to a diameter of the channel 320. The opening 344 is open to negative space formed between the arms 336, 340 to form an unobstructed path through which a bone pin and second hinge pin 380 may be received.

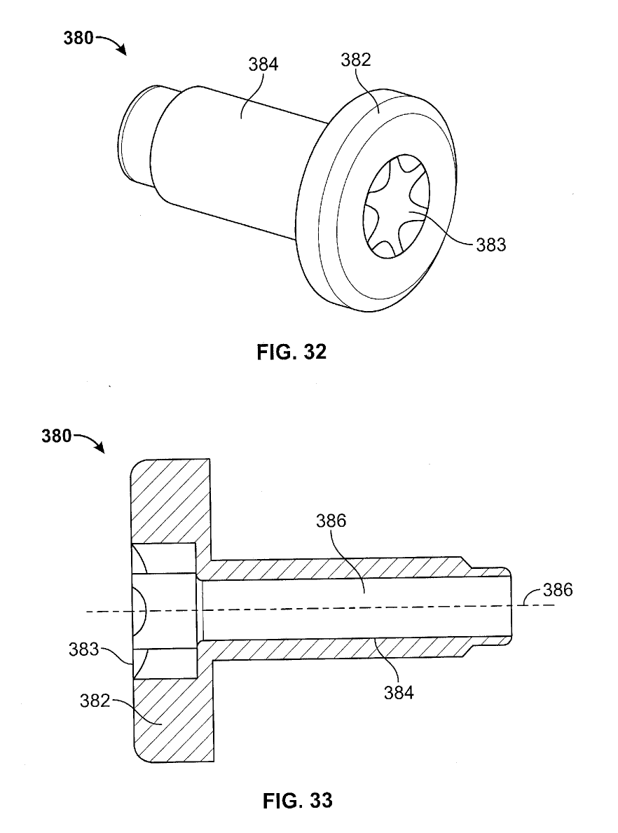

As shown in FIGS. 32-33, the second hinge pin 380 is substantially similar to the second hinge pin 180 and includes an increased diameter head 382 and an elongated shaft 384 extending therefrom. The head 382 includes a recess 383 or other mechanism to permit engagement thereof with a driving mechanism, as those skilled in the art will understand. An elongated channel 386 extends through the second hinge pin 380 to accommodate insertion of the bone pin therethrough. The shaft 384 may be threaded to permit threaded engagement of the second hinge pin 380 with the second rod 312, as will be described in greater detail with respect to the exemplary method below.

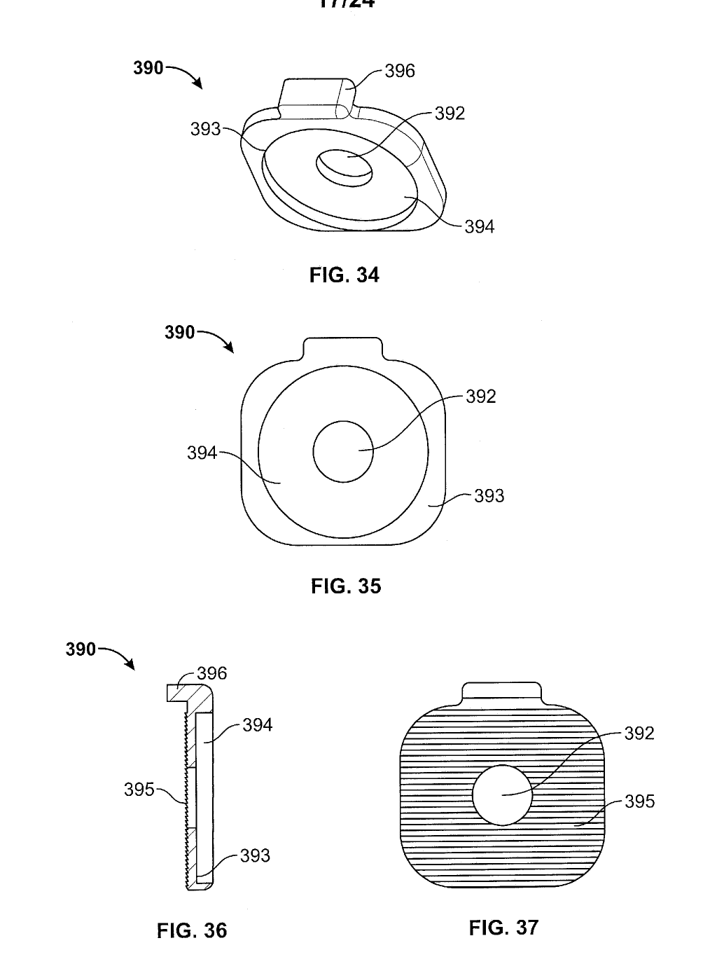

As shown in FIGS. 34-37, the device 300 further comprises a washer 390 substantially similar to the washer 190 except as noted below. The washer 390 includes a washer opening 392 extending therethrough and to align with the channel 320 and the opening 344 in an operative configuration. A lateral surface 393 of the washer 390 bordering the washer opening 392 includes a recessed portion 394 formed to house at least a portion of the head 382 therein. A medial surface 395 of the washer 390 includes a plurality of serrations 396 formed to interlock with the serrations 444, 464 of the first plate 430. The medial surface 395 further comprises a protruding tab 396 extending perpendicularly therefrom. The tab 396 is dimensioned to be received in the recess 448, 468 in an operative configuration, thus preventing any lateral movement of the washer 390 relative thereto. Similar to the washer 190, a length of the washer 390 is smaller than a length of the second portion 440 of the plate 402, thus permitting a surgeon or other user to selectively position the washer 390 therealong to conform to the requirements of a procedure.

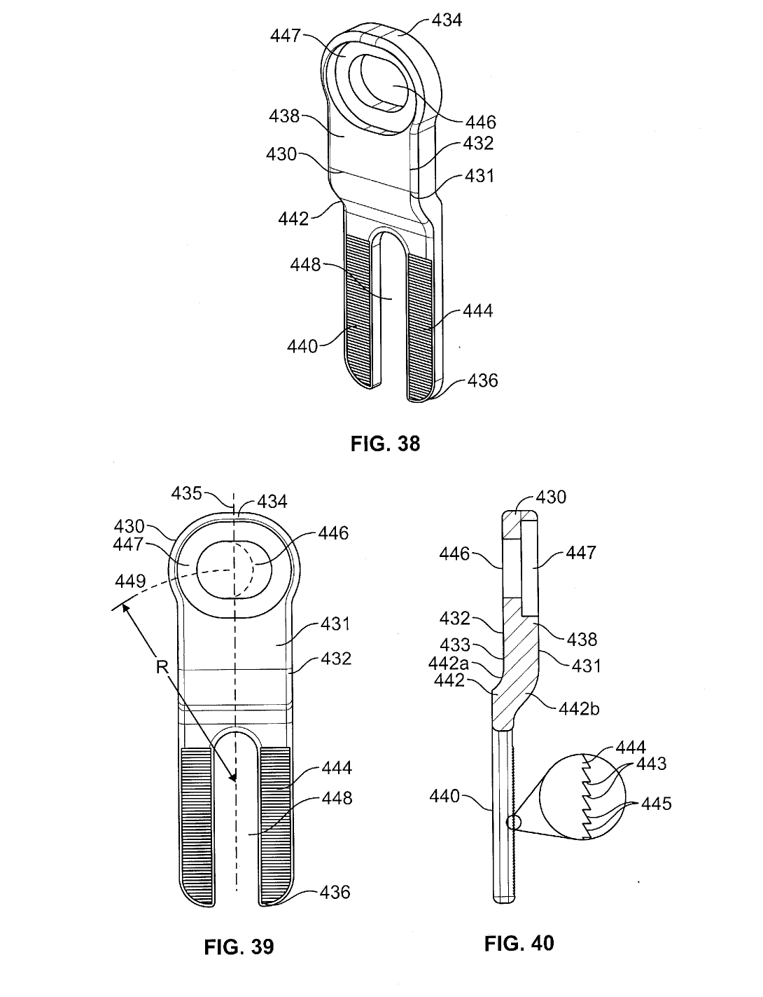

FIGS. 38-40 depict a first lateral plate 430 substantially similar to the first plate 130. The plate 430 includes an elongated plate body 432 extending from a first end 434 to a second end 436 along a longitudinal axis 435. The body 432 includes a first portion 438 having a first thickness and a second portion 440 having a second thickness smaller than the first thickness. The first and second portions 438, 440 are separated from one another by an angled portion 442. However, whereas the angled wall 142 of the first plate 130 is angled on only a lateral surface thereof, the angled portion 442 of the bone plate 430 includes first and second angled walls 442a, 442b provided on both lateral and medial surfaces 431, 433. A radius of curvature of the first angled wall 442a is smaller than a radius of curvature of the second angled wall 442b, as shown in FIG. 40. The angled portion 442 is formed such that the first and second portions 438, 440 of the first plate 430 are housed within two different planes extending parallel to one another. A length and thickness of each of the first and second portions 438, 440 as well as an angle of the angled portion 442 is selected to conform to the contour of the bone of the bone joint being corrected, as those skilled in the art will understand.

The first portion 438 of the first plate 430 further comprises a first plate hole 446. Whereas the first plate hole 146 is substantially circular, the first plate hole 446 is substantially oval, a cross-sectional shape thereof being formed by two overlapping portions, as shown in FIG. 39. In an exemplary embodiment, one of the overlapping shapes is a circle having a diameter of approximately 9 mm., although other dimensions may be used without deviating from the scope of the invention. The first plate hole 446 is elongated in a direction extending substantially perpendicular to the longitudinal axis 435 and is angled along an axis 449. Specifically, a radius of curvature R of the first plate hole 446 is selected such that the first plate 430 may slide over the axis 449. Furthermore, the oval shape of the first plate hole 446 allows for a partial sliding of the device 300 when implanted in a knee joint (e.g., during bending of the knee) to allow for a slightly increased range of movement of the joint. The angle of the axis 449 corresponds to an axis of in-situ rotation of the first rods 302 relative to respective first plate holes 446, 466. The design of the first plate 430 further aids in unloading of the knee joint so that forces normally experienced in the knee joint are distributed throughout the implant, thereby allowing a regeneration or healing of the cartilage tissue. Furthermore, this design increases mobility of the patient while minimizing discomfort and pain. The limited mobility offered by the implant of the invention is in contrast to conventional, external fixation systems which do not allow for any movement while worn. Rather, such systems must be removed to allow for movement. The exemplary system according to the invention allows for a limited range of movement without the need for removal, thus preserving the functioning of soft tissue at the knee joint. The radius of curvature R is taken from a midpoint of the first longitudinal recess 448 although any other placement may be used without deviating from the scope of the invention. A lateral end of the first plate hole 446 includes a beveled lip 447 to permit seating of the head 372 of the first hinge pin 370 therein.

The second portion 440 of the first plate 430 comprises the first longitudinal recess 448 extending therethrough and open to the second end 436. The first recess 448 extends parallel to the longitudinal axis 435. Serrations 444 on the lateral surface 431 of the second portion 440 are formed as a plurality of pointed teeth, each having a first wall 443 extending perpendicular to the longitudinal axis 435 and a second wall 445 at an angle of approximately 60 degrees relative to the first wall 443. The serrations 444 are formed to engage corresponding serrations 396 formed on the washer 390 which is positioned thereagainst in an operative configuration. In an exemplary embodiment, the serrations 444 are only provided over a predetermined length of the second portion 440 to coincide with a region which contacts the washer 390 in the operative configuration. In another embodiment, however, the serrations 444 may be provided over the entirety of the second portion 440, as with the device 100.

The second plate 450, depicted in greater detail in FIGS. 41-43, is substantially similar to first plate 430, except as noted below and is adapted for use on a medial side of a bone joint. Whereas the first and second plates 130, 150 of the device 100 may be used interchangeably, the first and second plates 430, 450 may only be used in the lateral and medial positions, respectively. Furthermore, the exemplary system 300 including the first and second plates 430, 450 and corresponding fixation means are formed to permit distraction of only one of the lateral and medial ends of the knee joint, based on the requirements for a particular patient. For example, if a surgeon determines that a condition of cartilage on one side of the knee joint is undamaged, a bone plate may be positioned on only the side of joint having damaged cartilage to only cause unloading of this side. The second plate 450 includes an elongated plate body 452 extending from a first end 454 to a second end 456 along a longitudinal axis 455. The body 452 includes a first portion 458 having a first thickness and a second portion 460 having a second thickness smaller than the first thickness, the first and second portions 458, 460 being separated from one another by an angled wall 462. In an exemplary embodiment, the angled wall 462 is provided on only a medial surface 451 of the second plate 450 while a lateral surface 453 is substantially planar, as shown in FIG. 43.

The first portion 458 of the second plate 450 further comprises a second plate hole 466 substantially similar to the first plate hole 446, as described in greater detail earlier. The second plate hole 466 is elongated in a direction extending substantially perpendicular to the longitudinal axis 455 and is angled along an axis 469. A radius of curvature R of the second plate hole 466 is selected such that the second plate 450 may be rotated about the axis 469. The radius of curvature R is taken from a midpoint of the second longitudinal recess 468 although any other placement may be used without deviating from the scope of the invention. A medial end of the second plate hole 466 includes a beveled lip 467 to permit seating of the head 372 of the first hinge pin 370 therein. The second portion 460 of the second plate 450 comprises a second longitudinal recess 468 extending therethrough and open to the second end 456. The second longitudinal recess 468 extends parallel to the longitudinal axis 455 and is formed substantially similar to the first longitudinal recess 448. Serrations 464 provided on the medial surface 451 of the second portion 460 are formed as a plurality of pointed teeth, each having a first wall 463 extending perpendicular to the longitudinal axis 455 and a second wall 465 extending at an angle of approximately 60 degrees relative to the first wall 463. As described in greater detail earlier, the serrations 464 are formed to engage corresponding serrations 396 of the washer 390.

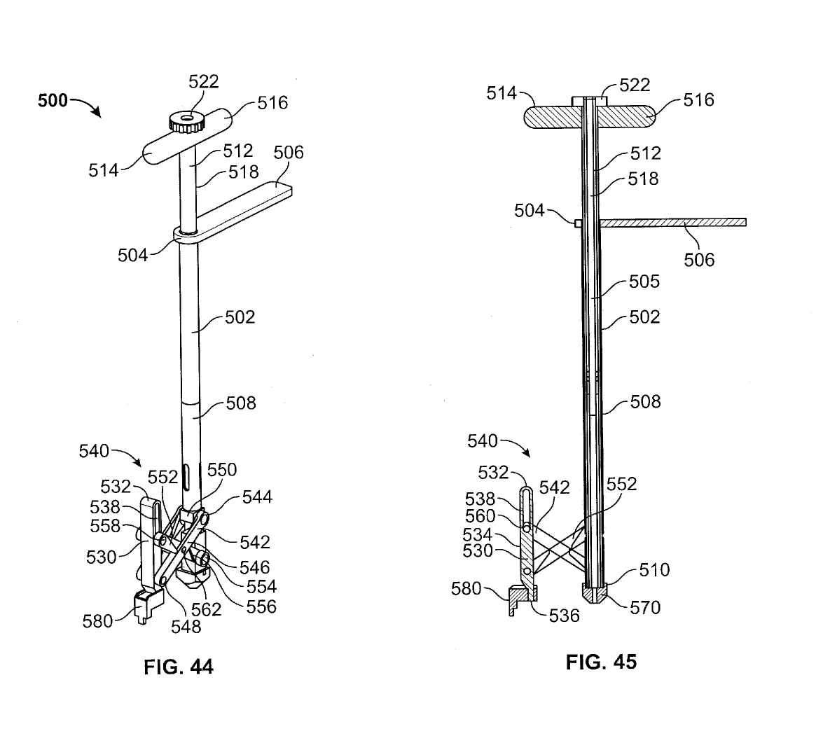

FIGS. 44-49 detail components of a distraction tool 500 according to the invention. The distraction tool 500 includes a first elongated cannulated rod 502 extending from a first end 504 having a first handle 506 and along an elongated shaft 508 to a distal end 510 associated with a scissor-like mechanism 540. A channel 505 extends though the first rod 502. The distraction tool 500 further comprises a second elongated cannulated rod 512 extending from a first end 514 having a second handle 516 and along an elongated shaft 518 to a distal end (not shown) received within the first rod 502. A locking screw 522 is provided at the first end 514 to lock a position of the first and second rods 502, 512 in an assembled configuration. The screw 522 may be removed from the tool 500 to permit disassembly of components thereof for sterilization. The first and second rods 502, 512 are oriented such that rotation of the second handle 516 causes a corresponding axial movement of the second rod 512 relative to the first rod 502. The device further comprises a third rod 530 extending from a first end 532 and along a shaft 534 to a second end 536. An opening 538 is formed through a first portion of the shaft 534, the opening permitting actuation of the scissor-mechanism 540 within a predetermined range, as will be described in greater detail hereinafter.

The scissor mechanism 540 includes first and second pairs of scissor-arms 542, 552 disposed on opposite sides of the tool 500. Each of the first scissor-arm extends from a first end 544 and along a shaft 546 to a second end 548 coupled to the third rod 530. The first end 544 is permanently coupled to a ring member 550 via, for example, a pin, rivet or screw. The ring member 550 is slidable over an outer wall of the first rod 502. The second end 538 is permanently coupled to the third rod 530 at a location adjacent to but spaced from the second end 536. The second scissor-arm 552 extends from a first end 554 and along a shaft 556 to a second end 558 coupled to the third rod 530. The first end 554 is permanently coupled to the outer wall of the first rod 502 via, for example, a pin, rivet or screw. The second end 558 includes a tab 560 extending transversely therefrom and into the opening 538 such that the tab 560 is axially slidable along a length of the opening 538. The first and second scissor-arms 542, 552 are further connected to one another about a pivot point 562. The second pair of scissor-arms 542, 552 formed on the opposing side of the tool 500 is substantially similar to the first pair of scissor-arms 542, 552 described above with the exception that no tab 560 is provided on the second scissor arm 552. Rather, the single tab 560 is connected to second ends 558 of both of the second scissor-arms 552, as can be seen in FIG. 44.

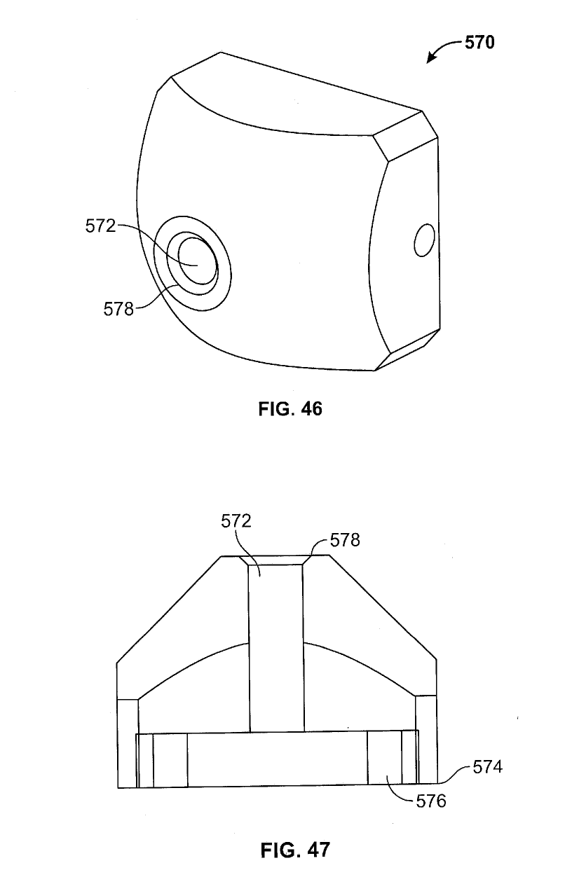

The first rod 502 further comprises an end cap 570 coupled to the second end 510, the end cap having an opening 572 aligning with the channel 505. A diameter of the opening 572 is selected to permit insertion of Kirschner wire 20′ therethrough. As shown in greater detail in FIGS. 46-47, the end cap 570 extends from a first end 574 having a recess 576 formed therein to a second end 578. The recess 576 is formed to receive the second end 510 of the first rod 502 with a friction fit. In another embodiment, the recess 576 and second end 510 may be threaded to aid in locking engagement therebetween.

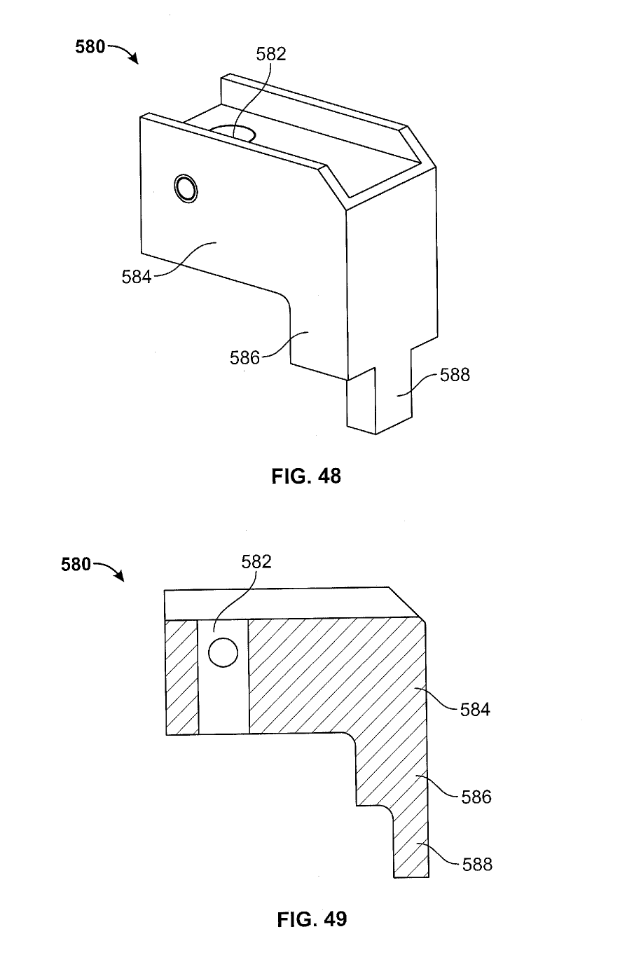

As shown in FIGS. 48-49, the third rod 530 further comprises a stepped foot 580 coupled to the second end 536. Specifically, the second end 536 may be formed with a reduced width relative to outlying portions of the third rod 530 and may include a substantially cylindrical shape. This cylindrical second end 536 may be received in a corresponding cylindrical opening 582 formed in the foot 580 and secured thereto via one of a friction-fit and a threaded engagement. The stepped foot 580 includes a substantially rectangular body 584 having a first stepped portion 586 extending transversely out of a first side thereof, the first stepped portion 586 having a width substantially equivalent to a width of the body 584 and a thickness smaller than a thickness of the body 584. A second stepped portion 588 extending transversely away from the first stepped portion 586 and having a width smaller than the width of the first stepped portion 586 and a thickness smaller than a thickness of the first stepped portion 586. In an operative configuration, the second stepped portion 588 is inserted into a gap 457, 477 formed after insertion of the washer 390 into the first and second recesses 448, 468 to aid in distraction of the knee joint, as will be described in greater detail hereinafter.

In accordance with an exemplary method according to the invention, a desired axis of rotation of the knee joint is determined. This determination may be achieved using a translucent template, as those skilled in the art will understand. Medial and lateral puncture incisions are formed in the area of the knee joint to permit a minimally invasive implantation of the device 100. Alternatively, a single incision traversing the knee joint can be performed to implant the device 300. A first Kirschner wire 20′ is then inserted into the femur 101 by means of a universal drilling machine along the axis of rotation of the femur 10, as described in greater detail earlier with respect to the device 100. An adjustable drill guide (not shown) is then adjusted to a position to guide a second Kirschner wire 30′ into the tibia 101′ such that the first and second Kirschner wires 20′, 30′ extend parallel to one another and are separated from one another by a space Z, as also described in greater detail earlier. The second Kirschner wire 30′ is positioned in the tibia 101′ by means of the drilling machine (not shown). The drill guide (not shown) is then removed. In a next exemplary step, the first rods 302 are hammered into lateral and medial ends of the femur such that the second ends 308 are aligned with an outer periphery of the bone, as shown in FIGS. 15 and 19. The first and second plates 430, 450 are then positioned over the first and second Kirschner wires 20′, 30′. First hinge pins 370 are then completely inserted through first and second plate holes 446, 466 of the first and second plates 430, 450 and into the first rods 302, as shown in FIGS. 15 and 19.

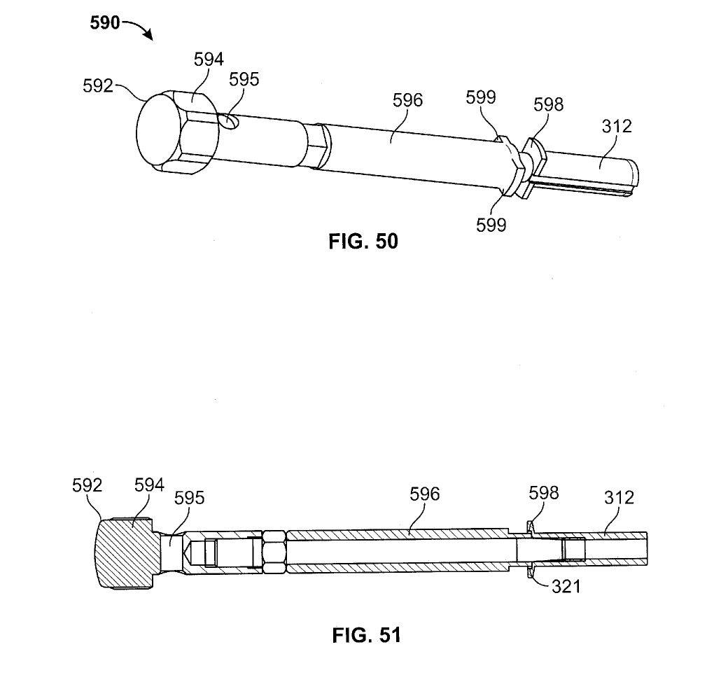

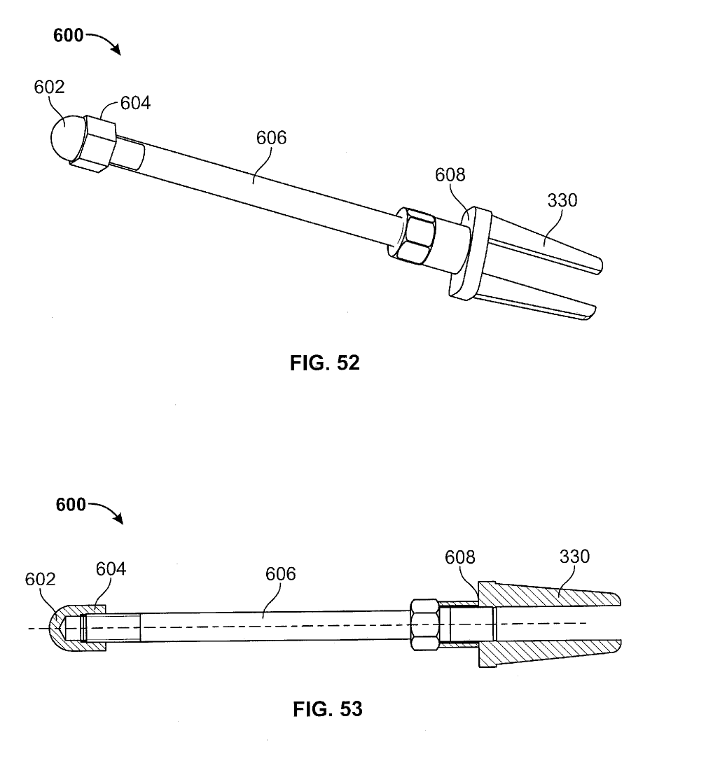

In a next exemplary step, the second rod 312 is inserted into the tibia via a first tibial insertion instrument 590. As shown in FIGS. 50-51, the first tibial insertion instrument 590 extends from a first end 592 including a head 594 and along an elongated shaft 596 to a second end 598, which is configured to engage the non-circular recess 321 of the second rod 312. The second end 598 is non-rotatably attached to the recesses 321 such that rotation of the first tibial insertion instrument tool 590 causes a corresponding rotation of the second rod 312, as will be described in greater detail later on. The first tibial insertion instrument 590 further comprises a pair of protrusions 599 disposed adjacent to the second end 598 and separated from one another by 180 degrees. The protrusions 599 serve as a visual indicator of a degree of rotation of the first tibial insertion instrument 590. In an exemplary configuration, the head 594 may be hammered to insert the second rod 312 into the tibia. Once the head 313 of the second rod 312 is positioned beyond the first and second longitudinal recesses 448, 468, respectively, an elongated bar (not shown) is inserted into the transverse opening 595. A user then applies rotational torque to the bar (not shown) to cause a corresponding rotation of the second rod 312 within the bone. The bar (not shown) is rotated by approximately 90 degrees under visual guidance of the protrusions 599 such that the flattenings 317 of the second rod 312 are no longer aligned with the first and second longitudinal recesses 448, 468. As those skilled in the art will understand, this configuration prevents the second rod 312 from moving out of the tibia in the implanted configuration. The blade 330 is then inserted through each of the first and second longitudinal recesses 448, 468 under the guidance of a second tibial insertion instrument 600, as shown in FIGS. 52-53. The second tibial insertion instrument 600 extends from a first end 602 including a head 604 and along an elongated shaft 606 to a second end 608, which is configured to engage the opening 344 extending into the blade 330. In an exemplary configuration, the head 604 may be hammered to insert the blade 330 into the tibia over the second rod 312, the arms 336, 340 being guided into the tibia along a path defined by the slots 319 of the second rod 312. The blade 330 is inserted into the tibia until the head 334 is seated within the one of the first ands second longitudinal recesses 448, 468, as shown in FIGS. 15 and 19. The washer 390 and second hinge pin 380 are then inserted through the first ands second longitudinal recesses 448, 468 such that the shaft 384 loosely engages the second rod 312.

The distraction tool 500 is then attached to the Kirschner wire 20′ and the gaps 457, 477 formed in the first and second plates 457, 477. Specifically, the Kirschner wire 20′ is received within the opening 572, extending into the channel 505 by any distance. The scissor mechanism 540 is then manipulated to cause a desired distraction of the knee joint, as described in greater detail earlier. This distraction causes the stepped foot 580 to engage a flat outer surface of the tab 396 of the washer 390 as the distraction tool 500 is widened. Once the first and second rods 302, 312 have been distracted to the desired spacing, the second hinge pins 380 are tightened to rigidly engage the first and second plates 430, 450 to the second rods 312. The distractor instrument 500 and Kirschner wires 20′, 30′ are then removed and incisions closed. The above-recited method is substantially the same in the case where only one of the first and second plates 430, 450 is to be secured to the knee joint.

In another exemplary method according to the invention, one or both of the first rods 302 and one or both sets of second rods 312 and blades 330 may be inserted into bones 101, 101′ prior to positioning of the first and second plates 430, 450 thereover. This method is substantially the same as disclosed above. Specifically, after drilling holes through each of the femur 101 and tibia 101′, the Kirschner wires 20′, 30′ are inserted into the drilled holes. The first rods 302 are inserted into the femur 101. As those skilled in the art will understand, if distraction is only required for one side of the knee joint, only one first rod 302 is inserted into the target side. The second rod 312 is then inserted into the tibia 101′, followed by the blade 330. In an exemplary embodiment, the blade 330 may be inserted such that the arms 336, 340 are aligned with longitudinal slots 319, as described in greater detail earlier and an elongated portion thereof having the length L is not aligned with the first ands second longitudinal recesses 448, 468, thus preventing withdrawal thereof from the first and second plates 430, 450. One or both of the first and second plates 430, 450 is then inserted over the knee joint and successively fixed to the femur 101 and tibia 101′, as described in greater detail earlier. The knee joint is then distracted and the washer 390 tightened.

It will be appreciated by those skilled in the art that various modifications and alterations of the invention can be made without departing from the broad scope of the appended claims. Some of these have been discussed above and others will be apparent to those skilled in the art.

What is claimed is:

1. A device for bone joint distraction, comprising:
a first bone plate for insertion on a first side of the bone joint;
a second bone plate for insertion on a second side of the bone joint;
a first elongated rod insertable into the bone joint, wherein the first bone plate is non-rotatably coupled to the first rod; and
a second elongated rod insertable into the bone joint, wherein the second bone plate is rotatably coupled to the second rod, the first and second bone plates being movable relative to one another within a predetermined range of movement in a plane including longitudinal axes of the first and second rods to distract the joint.

2. The device of claim 1, further comprising a first hinge pin insertable through a first plate hole in the first bone plate and into the first elongated rod.

3. The device of claim 2, wherein the first plate hole is circular.

4. The device of claim 1, wherein the first plate hole is oval and elongated along an arc of rotation of the joint.

5. The device of claim 1, wherein a length of each of the first and second elongated rods is selected to match a thickness of a portion of a bone through which it is inserted.

6. The device of claim 1, wherein a length of each of the first and second elongated rods is smaller than a thickness of a bone through which it is inserted, the length being smaller than half of the thickness of the bone.

7. The device of claim 6, further comprising:
an additional first elongated rod, wherein the first elongated rod is inserted into a first end of a hole formed in a first bone and the additional first elongated rod is inserted into an opposing end of the hole formed in the first bone; and
an additional second elongated rod, wherein the second elongated rod is inserted into a first end of a hole formed in a second bone and the additional second elongated rod is inserted into an opposing end of the hole formed in the second bone.

8. The device of claim 2, further comprising:
a second hinge pin insertable through a first elongated recess formed through the first bone plate and into the second elongated rod, wherein the first elongated recess is elongated in a direction extending parallel to a longitudinal axis of the first bone plate; and
a washer positioned between the second hinge pin and the first bone plate.

9. The device of claim 8, wherein the first bone plate included a bone-contacting surface and a non-bone contacting surface.

10. The device of claim 9, wherein a region of the non-bone contacting surface adjacent the first elongated recess includes a plurality of serrations formed to interlock with a corresponding plurality of serrations formed on the washer.

11. The device of claim 8, wherein the non-bone contacting surface of the first bone plate includes a curvature selected to conform to an anatomy of the bone joint.

12. The device of claim 1, wherein the second elongated rod includes an increased diameter head having first and second cutouts on opposing sides thereof, the cutouts being dimensioned to permit insertion of the second elongated rod through the first elongated recess in only a predetermined orientation.

13. The device of claim 12, further comprising a blade positionable over the second elongated rod, the blade having first and second arms receivable over corresponding slots formed on an outer wall of the second elongated rod.

14. The device of claim 1, wherein an outer wall of the first elongated rod includes a plurality of ribs extending radially therefrom.

15. The device of claim 1, wherein the first and second bone plates are interchangeably usable.

16. A method for distracting a bone joint, comprising:
inserting a first elongated rod into a first bone comprising a bone joint along a path defined by a first bone pin;
inserting a second elongated rod into a second bone comprising the bone joint along a path defined by a second bone pin;
positioning a first bone plate over a lateral wall of the bone joint and a second bone plate over a medial wall of the bone joint;
inserting a first hinge pin into a first plate hole of the first bone plate and into the first bone and inserting a second hinge pin into a second plate hole of the second bone plate and into the first bone;
inserting a third hinge pin into a first longitudinal recess of the first bone plate and into the second bone and inserting a fourth hinge pin into a second longitudinal recess of the second bone plate and into the second bone;
distracting the joint by changing a spacing between the first and second elongated rods to a desired spacing relative to one another; and
fixing the third and fourth hinge pins to lock a position of the first and second rods relative to one another when the desired distraction has been achieved.

17. The method of claim 16, further comprising:
positioning free ends of the first and second bone pins on a distraction tool: and
manipulating the distraction tool to cause a corresponding movement of the first and second rods relative to one another.

18. The method of claim 16, further comprising:
rotating the second rod within the second bone to cause an increased diameter head to rotate with respect to the first longitudinal recess; and
inserting a blade over the second elongated rod, the blade having first and second arms formed to align with corresponding slots formed on the second rod.

19. The method of claim 16, further comprising positioning a spacer between the second hinge pin and the first bone plate, the spacer having a plurality of serrations formed to interlock with corresponding serrations of the first bone plate in a fixed configuration.

20. An instrument for displacement of first and second bones of a bone joint, comprising:
a first implant holder adapted to hold a first pin inserted into the first bone of the bone joint;
a second implant holder adapted to hold the second bone of the bone joint, the second implant holder being pivotally connected to the first implant holder; and
a displacing arrangement movable to cause a controlled movement of the first and second implant holders relative to one another, the controller movement causing a corresponding movement of the first and second bones relative to one another.

21. The instrument of claim 20, wherein the first and second implant holders are connected to one another about first and second pivot points.

22. The instrument of claim 20, wherein the displacing arrangement comprises a first bolt threadably received over a bar connecting the first and second implant holders to one another, wherein threaded movement of the first bolt in a first direction along the bar causes the first and second implant holders to move toward one another and wherein movement of the first bolt in a second direction permits movement of the first and second implant holders away from one another.

23. The instrument of claim 22, further comprising a second bolt threadedly positioned over the bar, wherein movement of the second bolt in the second direction causes the first and second implant holders to move away from one another.

24. The instrument of claim 20, wherein the second implant holder is adapted to hold a second pin inserted into the second bone.

25. The instrument of claim 20, wherein the second implant holder is adapted to hold a plate positioned over an outer surface of the bone.

26. The instrument of claim 25, wherein the displacing arrangement comprises:
a first cannulated rod extending from a first end to a second end, the first end including a first handle and the second end including the first implant holder; and
a second cannulated rod extending from a first end having a second handle to a second end received within the first cannulated rod;
wherein rotation of the second handle causes a corresponding axial movement of the second cannulated rod relative to the first cannulated rod, the axial movement causing a corresponding lateral movement of the first and second implant holders relative to one another.

27. The instrument of claim 26, wherein the second implant holder is formed as a stepped block having an abutment formed at a distal end thereof to engage the plate.

28. The instrument of claim 27, wherein the stepped block is further attached to an elongated rod, the rod being connected to the first and second cannulated rods via a scissor-mechanism.

29. The instrument of claim 26, further comprising a locking screw fixing the first cannulated rod to the second cannulated rod.
